(19) United States
(12) Patent Application Publication
(10) Pub. No.: US 2024/0093192 A1
(43) Pub. Date: Mar. 21, 2024

(54) ANTISENSE OLIGONUCLEOTIDES INCREASING FOXG1 EXPRESSION

(71) Applicant: EliGab Tx LLC, Manhasset, NY (US)

(72) Inventors: Scott REICH, Manhasset, NY (US); Hans-Peter Vornlocher, Manhasset, NY (US); Anke Geick, Manhasset, NY (US); Brian Bettencourt, Manhasset, NY (US)

(21) Appl. No.: 18/336,617

(22) Filed: Jun. 16, 2023

Related U.S. Application Data
(63) Continuation of application No. PCT/US22/15815, filed on Feb. 9, 2022.
(60) Provisional application No. 63/148,030, filed on Feb. 10, 2021.
(60) Provisional application No. 63/224,314, filed on Jul. 21, 2021.

(51) Int. Cl. C12N 15/113

(52) U.S. Cl. CPC C12N 15/113; C12N 2310/113; C12N 2310/315; C12N 2310/321; C12N 2310/341

(57) ABSTRACT

Provided herein are compositions and methods for treating and/or ameliorating the FOXG1 syndromes or the symptoms associated therewith. The compositions and methods disclosed herein utilize antisense oligonucleotides that target long non-coding RNAs (lncRNAs) to increase FOXG1 expression in a cell, thereby restoring FOXG1 function.

### CROSS-REFERENCE

This application claims the benefit of U.S. Provisional Patent Application No. 63/148,030, filed Feb. 10, 2021, and this application claims the benefit of U.S. Provisional Patent Application No. 63/224,314, filed Jul. 21, 2021, which are incorporated herein by reference in their entirety.

### BACKGROUND

FOXG1 syndrome is a rare neurodevelopmental disorder associated with heterozygous variants in the forkhead box G1 (FOXG1) gene and is characterized by impaired neurological development and/or altered brain physiology. Observed phenotypes of FOXG1 syndrome primarily include a particular pattern of structural alterations in the brain resulting from inherited de novo mutations in the FOXG1 gene. Such structural alterations include a thin or underdeveloped corpus callosum that connects between the right and left hemispheres of the brain, reduced sulci and gyri formation on the surface of the brain, and/or a reduced amount of white matter. FOXG1 syndrome affects most aspects of development in children and the main clinical features observed in association with FOXG1 variants comprise impairment of postnatal growth, primary (congenital) or secondary (postnatal) microcephaly, severe intellectual disability with absent speech development, epilepsy, stereotypies and dyskinesia, abnormal sleep patterns, unexplained episodes of crying, gastroesophageal reflux, and recurrent aspiration.

### SUMMARY

Provided herein are compositions and methods for treating and/or ameliorating FOXG1 syndrome or the symptoms associated therewith. The compositions and methods described herein utilize antisense oligonucleotides that target long non-coding RNAs (lncRNAs) to increase FOXG1 expression. In certain instances, the targeted long non-coding RNAs (lncRNAs) down regulate FOXG1 expression (e.g. mRNA or protein), wherein the antisense oligonucleotides (ASOs) thereby prevent or inhibit or reduce lncRNA-mediated down-regulation of FOXG1 expression. The ability to restore or increase functional FOXG1 expression in cells provides a foundation for the treatment of FOXG1 syndrome or alleviating symptoms associated therewith. The compositions and methods described herein are, in part, based on the discovery that FOXG1 expression can be increased by targeting long non-coding RNAs (lncRNAs) with antisense oligonucleotides. Accordingly, the present disclosure (i) provides that FOXG1 expression can be increased by targeting long non-coding RNAs (lncRNAs) with antisense oligonucleotides, and (ii) provides assays and methods for the identification of antisense oligonucleotides that increase FOXG1 expression by targeting long non-coding RNAs (lncRNAs).

Provided herein are antisense oligonucleotides (ASOs), comprising a sequence that hybridizes to a target nucleic acid sequence of a long non-coding RNA (lncRNA). In some embodiments, the lncRNA regulates expression of FOXG1. In some embodiments, the lncRNA reduces expression of FOXG1 messenger RNA. In some embodiments, the lncRNA reduces transcription of FOXG1 messenger RNA molecule. In some embodiments, the lncRNA reduces expression of FOXG1 protein. In some embodiments, the lncRNA reduces translation of a FOXG1 protein molecule.

In some embodiments, provided is an antisense oligonucleotide of any of the preceding embodiments, wherein the antisense oligonucleotide comprises a modification. In some embodiments, provided is an antisense oligonucleotide of any of the preceding embodiments, wherein the modification comprises a modified inter-nucleoside linker, a modified nucleoside, or a combination thereof. In some embodiments, provided is an antisense oligonucleotide of any of the preceding embodiments, wherein the antisense oligonucleotide comprises a modified inter-nucleoside linkage. In some embodiments, provided is an antisense oligonucleotide of any of the preceding embodiments, wherein the modified inter-nucleoside linkage is a phosphorothioate inter-nucleoside linkage. In some embodiments, provided is an antisense oligonucleotide of any of the preceding embodiments, wherein the antisense oligonucleotide comprises a phosphodiester inter-nucleoside linkage. In some embodiments, provided is an antisense oligonucleotide of any of the preceding embodiments, wherein the antisense oligonucleotide comprises a modified nucleoside. In some embodiments, provided is an antisense oligonucleotide of any of the preceding embodiments, wherein the modified nucleoside comprises a modified sugar. In some embodiments, provided is an antisense oligonucleotide of any of the preceding embodiments, wherein the modified sugar is a bicyclic sugar. In some embodiments, provided is an antisense oligonucleotide of any of the preceding embodiments, wherein the modified sugar comprises a 2′-O-methoxyethyl group.

In some embodiments, provided is an antisense oligonucleotide of any of the preceding embodiments, wherein the sequence is complementary to the target nucleic acid sequence of a long non-coding RNA (lncRNA). In some embodiments, provided is an antisense oligonucleotide of any of the preceding embodiments, wherein the long non-coding RNA (lncRNA) is located within 1 kilobases (kb), 2 kb, 5 kb, 8 kb, or 10 kb of a gene encoding FOXG1. In some embodiments, provided is an antisense oligonucleotide of any of the preceding embodiments, wherein the long non-coding RNA (lncRNA) is FOXG1-AS1 (https://www.ncbi.nlm.nih.gov/gene/103695363), long non-protein coding RNA 1551 (LINC01151); see https://www.ncbi.nlm.nih.gov/gene/387978), long intergenic non-protein coding RNA 2282 (LINC02282); see https://www.ncbi.nlm.nih.gov/gene/105370424), or a combination thereof.

In some embodiments, provided is an antisense oligonucleotide of any of the preceding embodiments, wherein the sequence comprises a nucleobase sequence as set forth in any one of Table 3. In some embodiments, provided is an antisense oligonucleotide of any of the preceding embodiments, wherein the antisense oligonucleotide comprises a nucleobase sequence as set forth in any one of Table 4. In some embodiments, provided is an antisense oligonucleotide of any of the preceding embodiments, wherein the antisense oligonucleotide hybridizes to the target nucleic acid sequence comprising or adjacent to any one or more of the positions provided in Table 3. In some embodiments, provided is an antisense oligonucleotide of any of the preceding embodiments, wherein adjacent to any one or more of the positions provided in Table 3 comprises base positions within 20, 40, 50, 75, 100, or 150 base positions 5′ and/or 3′. In some embodiments, provided is an antisense oligonucleotide of any of the preceding embodiments, wherein the antisense oligonucleotide hybridizes to the target nucleic acid sequence comprising or adjacent to any one or more of the positions provided in Table 4, In some embodiments, provided is an antisense oligonucleotide of any of the preceding embodiments, wherein adjacent to any one or more of the positions provided in Table 4 comprises base positions within 20, 40, 50, 75, 100, or 150 base positions 5′ and/or 3′.

In some embodiments, provided is an antisense oligonucleotide of any of the preceding embodiments, wherein hybridization of the sequence of the antisense oligonucleotide to the target nucleic acid sequence increases degradation of the lncRNA. In some embodiments, provided is an antisense oligonucleotide of any of the preceding embodiments, wherein hybridization of the sequence of the antisense oligonucleotide to the target nucleic acid sequence increases expression of FOXG1. In some embodiments, provided is an antisense oligonucleotide of any of the preceding embodiments, wherein expression of FOXG1 is mRNA expression. In some embodiments, provided is an antisense oligonucleotide of any of the preceding embodiments, wherein expression of FOXG1 is protein expression. A composition comprising one or more of the antisense oligonucleotides of any of the preceding embodiments. A pharmaceutical composition comprising the antisense oligonucleotide of any of the preceding embodiments 3 and a pharmaceutically acceptable carrier or diluent.

Further provided are methods of modulating expression of FOXG1 in a cell, comprising contacting the cell with a composition comprising an antisense oligonucleotide that hybridizes to a target nucleic acid sequence of a long non-coding RNA (lncRNA). Also provided are methods of treating or ameliorating a FOXG1 disease or disorder in an individual having, or at risk of having, the FOXG1 disease or disorder, comprising administering to the individual an antisense oligonucleotide, wherein the antisense oligonucleotide comprises a sequence that hybridizes to a target nucleic acid sequence of a long non-coding RNA (lncRNA).

In some embodiments, provided is a method of any of the preceding embodiments, wherein the cell is a located in a brain of an individual. In some embodiments, provided is a method of any of the preceding embodiments, wherein the individual is a human. In some embodiments, provided is a method of any of the preceding embodiments, wherein the individual comprises reduced FOXG1 expression or a FOXG1 deficiency. In some embodiments, provided is a method of any of the preceding embodiments, wherein the individual has a FOXG1 disease or disorder. In some embodiments, provided is a method of any of the preceding embodiments, wherein the FOXG1 disease or disorder is FOXG1 syndrome.

In some embodiments, provided is a method of any of the preceding embodiments, wherein the antisense oligonucleotide comprises a sequence that is complementary to the target nucleic acid sequence of a long non-coding RNA (lncRNA).

In some embodiments, provided is a method of any of the preceding embodiments, wherein the long non-coding RNA (lncRNA) is located within 1 kilobases (kb), 2 kb, 5 kb, 8 kb, or 10 kb of a gene encoding FOXG1. In some embodiments, provided is a method of any of the preceding embodiments, wherein the long non-coding RNA (lncRNA) is FOXG1-AS1, long non-protein coding RNA 1551 (LINC01151), long intergenic non-protein coding RNA 2282 (LINC02282), or a combination thereof.

In some embodiments, provided is a method of any of the preceding embodiments, wherein the antisense oligonucleotide comprises a nucleobase sequence as set forth in any one of Table 3. In some embodiments, provided is a method of any of the preceding embodiments, wherein the antisense oligonucleotide comprises a nucleobase sequence as set forth in any one of Table 4. In some embodiments, provided is a method of any of the preceding embodiments, wherein the antisense oligonucleotide hybridizes to the target nucleic acid sequence comprising or adjacent to any one or more of the positions provided in Table 3. In some embodiments, provided is a method of any of the preceding embodiments, wherein adjacent to any one or more of the positions provided in Table 3 comprises base positions within 20, 40, 50, 75, 100, or 150 base positions 5′ and/or 3′. In some embodiments, provided is a method of any of the preceding embodiments, wherein the antisense oligonucleotide hybridizes to the target nucleic acid sequence comprising or adjacent to any one or more of the positions provided in Table 4, In some embodiments, provided is a method of any of the preceding embodiments, wherein adjacent to any one or more of the positions provided in Table 4 comprises base positions within 20, 40, 50, 75, 100, or 150 base positions 5′ and/or 3′.

In some embodiments, provided is a method of any of the preceding embodiments, wherein hybridization of the antisense oligonucleotide to the target nucleic acid sequence increases degradation of the lncRNA. In some embodiments, provided is a method of any of the preceding embodiments, wherein hybridization of the antisense oligonucleotide to the target nucleic acid sequence increases expression of FOXG1. In some embodiments, provided is a method of any of the preceding embodiments, wherein expression of FOXG1 is mRNA expression. In some embodiments, provided is a method of any of the preceding embodiments, expression of FOXG1 is protein expression.

In some embodiments, provided is a method of any of the preceding embodiments, wherein the antisense oligonucleotide is configured as a gapmer. In some embodiments, provided is a method of any of the preceding embodiments, wherein the antisense oligonucleotide comprises at least one modified inter-nucleoside linkage. In some embodiments, provided is a method of any of the preceding embodiments, wherein the modified inter-nucleoside linkage is a phosphorothioate inter-nucleoside linkage. In some embodiments, provided is a method of any of the preceding embodiments, wherein the antisense oligonucleotide comprises at least one phosphodiester inter-nucleoside linkage. In some embodiments, provided is a method of any of the preceding embodiments, wherein the antisense oligonucleotide comprises a modified nucleoside. In some embodiments, provided is a method of any of the preceding embodiments, the modified nucleoside comprises a modified sugar. In some embodiments, provided is a method of any of the preceding embodiments, the modified sugar is a bicyclic sugar. In some embodiments, provided is a method of any of the preceding embodiments, wherein the modified sugar comprises a 2′-O-methoxyethyl group.

In some embodiments, provided is a method of any of the preceding embodiments, wherein modulating expression comprises increasing expression of a FOXG1 protein in the cell.

In some embodiments, provided is a method of any of the preceding embodiments, wherein modulating expression comprises increasing translation of a FOXG1 protein in the cell.

In some embodiments, provided is a method of any of the preceding embodiments, wherein the antisense oligonucleotide is administered to the individual by intrathecal injection, intracerebroventricular injection, inhalation, parenteral injection or infusion, or orally.

Also provided herein are gapmer antisense oligonucleotides comprising a sequence that hybridizes to a target nucleic acid sequence of a long non-coding RNA (lncRNA), wherein the lncRNA reduces expression of FOXG1. In some embodiments, expression of FOXG1 is measured by FOXG1 mRNA expression. In some embodiments, expression of FOXG1 is measured by FOXG1 protein expression.

In some embodiments, the antisense oligonucleotide comprises a modification. In some embodiments, the modification comprises a modified inter-nucleoside linker, a modified nucleoside, or a combination thereof. In some embodiments, the antisense oligonucleotide comprises the modified inter-nucleoside linkage. In some embodiments, the sequence comprises a nucleobase sequence as set forth in any one of Table 3. In some embodiments, the antisense oligonucleotide comprises a nucleobase sequence as set forth in any one of Table 4. In some embodiments, the target nucleic acid sequence comprises one or more nucleobases complementary to a sequence selected from Table 3. In some embodiments, the target nucleic acid sequence comprises one or more nucleobases within or adjacent to any one of the reference positions selected from Table 3. In some embodiments, the target nucleic acid sequence comprises one or more nucleobases complementary to a sequence selected from Table 4. In some embodiments, the target nucleic acid sequence comprises one or more nucleobases within or adjacent to any one of the reference positions selected from Table 4.

In some embodiments, hybridization of the antisense oligonucleotide increases FOXG1 expression in a cell. In some embodiments, the FOXG1 expression is FOXG1 mRNA expression. In some embodiments, the FOXG1 mRNA expression is measured by a probe based quantification assay. In some embodiments, the long non-coding RNA (lncRNA) is FOXG1-AS1, long intergenic non-protein coding RNA 1551, long intergenic non-protein coding RNA 2282 (LINC02282), or a combination thereof

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a diagram of a FOXG1 transcript.

FIGS. 2A, 2B, and 2C show gapmer antisense oligonucleotides (ASOs) that target long non-coding RNAs and increase FOXG1 expression.

### DETAILED DESCRIPTION

Deletions or mutations in a single allele of the forkhead box G1 (FOXG1) gene cause FOXG1 syndrome. FOXG1 syndrome is a rare disease characterized by developmental delay, severe intellectual disability, epilepsy, absent language, and dyskinesis. Hallmarks of altered brain physiologies associated with FOXG1 syndrome include cortical atrophy and agenesis of the corpus callosum. The FOXG1 gene/protein is a member of the forkhead transcription factor family and is expressed specifically in neural progenitor cells of the forebrain. The FOXG1 gene is composed of one coding exon and notably, the location or type of FOXG1 mutation can be associated with or indicative of clinical severity.

The FOXG1 protein plays an important role in brain development, particularly in a region of the embryonic brain known as the telencephalon. The telencephalon ultimately develops into several critical structures, including the largest part of the brain (i.e. cerebrum), which controls most voluntary activity, language, sensory perception, learning, and memory. A shortage of functional FOXG1 protein, as observed in individuals having mutations or deletions in a single FOXG1 allele (i.e. heterozygous individuals), disrupts normal brain patterning and development.

Expression of a target get can regulated by long non-coding ribonucleic acids (lncRNAs) at multiple levels. For example, by interacting with DNA, RNA and proteins, lncRNAs can modulate the transcription of neighboring and distant genes, and affect RNA splicing, stability and translation.

Accordingly, described herein are compositions and methods of modulation the status, activity, or expression of long intervening (which includes both intronic and intergenic) non-coding RNAs (lncRNAs) in a cell, tissue or organism. Also provided are compositions and methods for treating pathological conditions and diseases in a mammal caused by or modulated by the regulatory, structural, catalytic or signaling properties of a lncRNA. Accordingly, disclosed herein are compositions and methods useful for increasing an amount of functional FOXG1 (e.g. FOXG1 protein or FOXG1 messenger ribonucleic acid (mRNA)) in a cell having a shortage of functional FOXG1. Such compositions and methods are useful in their application for treating individual having a FOXG1-related disease or disorder wherein the lack or shortage of functional FOXG1 protein can be remedied. In order to achieve an increase of FOXG1 expression in cells or in an individual, antisense oligonucleotides targeting lncRNAs are used.

### Antisense Oligonucleotides

Antisense oligonucleotides (ASOs) are small (˜18-30 nucleotides), synthetic, single-stranded nucleic acid polymers that can be employed to modulate gene expression by various mechanisms. Antisense oligonucleotides (ASOs) can be subdivided into two major categories: RNase H competent and steric block. For RNase H competent antisense oligonucleotides, the endogenous RNase H enzyme recognizes RNA-DNA heteroduplex substrates that are formed when antisense oligonucleotides bind to their cognate mRNA transcripts to catalyze the degradation of RNA. Steric block oligonucleotides are antisense oligonucleotides (ASOs) that are designed to bind to target transcripts with high affinity but do not induce target transcript degradation.

In order to achieve effective targeting of a lncRNA, the antisense oligonucleotides (ASOs) describe herein hybridize to a target nucleic acid sequence of a long non-coding RNA (lncRNA). In certain instances, a lncRNA generally can be defined as an RNA molecule having great than about 200 nucleotides, wherein the RNA molecule does not encode for a protein sequence or translated protein sequence or translatable protein sequence. In certain instances, the lncRNA is transcribed from an intergene region or intraintronic region. In some embodiments, the lncRNA comprises greater than about 200 kilobases (kb), 400 kb, 500 kb, 1000 kb, 2000 kb.

lncRNAs can regulate FOXG1 through a one or more various or different mechanisms. In some embodiments, the lncRNA reduces expression of FOXG1 messenger RNA. In some embodiments, the lncRNA reduces transcription of FOXG1 messenger RNA molecule. In some embodiments, wherein the lncRNA reduces expression of FOXG1 protein. In some embodiments, he lncRNA reduces translation of a FOXG1 protein molecule.

Targeting (e.g. hybridization) to a lncRNA, in some embodiments, disclosed herein are antisense nucleotides (ASOs) comprising a sequence complementary or substantially complementary (e.g. having at least 70%, 80%, 90, 95%, or 100% sequence identity) to a target nucleic acid sequence of a long non-coding RNA (lncRNA). In some embodiments, the sequence is complementary to the target nucleic acid sequence of a long non-coding RNA (lncRNA). In some embodiments, the long non-coding RNA (lncRNA) is FOXG1-AS1, long non-protein coding RNA 1551 (LINC01151), long intergenic non-protein coding RNA 2282 (LINC02282), or a combination thereof. In some embodiments, the sequence comprises a nucleobase sequence as set forth in any one of SEQ ID NOs: 1-288.

In some embodiments, the sequence is complementary to the target nucleic acid sequence at or adjacent (e.g., surrounding) to positions 200-350 or 375-525 of long non-coding RNA (lncRNA) FOXG1-AS1 (e.g., reference sequence NR_125758.1). In some embodiments, the antisense oligonucleotide hybridizes to a target nucleic acid sequence at or adjacent (e.g., surrounding) to positions 200-350 or 375-525 of long non-coding RNA (lncRNA) FOXG1-AS1. In certain embodiments, the antisense oligonucleotide hybridizes to a target nucleic acid sequence at or adjacent (e.g., surrounding) to the positions provided in Table 3 or 4 for long non-coding RNA (lncRNA) FOXG1-AS1. In certain embodiments, the antisense oligonucleotide hybridizes to a target nucleic acid sequence adjacent (e.g., surrounding) to the positions provided in Table 3 for long non-coding RNA (lncRNA) FOXG1-AS1, wherein adjacent to or surrounding includes base positions within 20, 40, 50, 75, 100, or 150 base positions 5′ and/or 3′. In certain embodiments, the antisense oligonucleotide hybridizes to a target nucleic acid sequence adjacent (e.g., surrounding) to the positions provided in Table 4 for long non-coding RNA (lncRNA) FOXG1-AS1, wherein adjacent to or surrounding includes base positions within 20, 40, 50, 75, 100, or 150 base positions 5′ and/or 3′. In certain embodiments, the adjacent to or surrounding base positions are within 20 base positions 5′ and/or 3′. In certain embodiments, the adjacent to or surrounding base positions are within 40 base positions 5′ and/or 3′. In certain embodiments, the adjacent to or surrounding base positions are within 50 base positions 5′ and/or 3′. In certain embodiments, the adjacent to or surrounding base positions are within 75 base positions 5′ and/or 3′. In certain embodiments, the adjacent to or surrounding base positions are within 100 base positions 5′ and/or 3′. In certain embodiments, the adjacent to or surrounding base positions are within 150 base positions 5′ and/or 3′.

In some embodiments, the sequence is complementary to the target nucleic acid sequence at or adjacent (e.g., surrounding) to positions 950-1150 or 1450-1650 or 2150-2350 or 3450-3730 of long non-coding RNA (lncRNA) LINC01551 (e.g., reference sequence NR_026732.1 and NR_026731.1—merged exons). In some embodiments, the antisense oligonucleotide hybridizes to a target nucleic acid sequence at or adjacent (e.g., surrounding) to positions 950-1150 or 1450-1650 or 2150-2350 or 3450-3730 of long non-coding RNA (lncRNA) LINC01551. In certain embodiments, the antisense oligonucleotide hybridizes to a target nucleic acid sequence at or adjacent (e.g., surrounding) to the positions provided in Table 3 or 4 for long non-coding RNA (lncRNA) LINC01551. In certain embodiments, the antisense oligonucleotide hybridizes to a target nucleic acid sequence adjacent (e.g., surrounding) to the positions provided in Table 3 for long non-coding RNA (lncRNA) LINC01551, wherein adjacent to or surrounding includes base positions within 20, 40, 50, 75, 100, or 150 base positions 5′ and/or 3′. In certain embodiments, the antisense oligonucleotide hybridizes to a target nucleic acid sequence adjacent (e.g., surrounding) to the positions provided in Table 4 for long non-coding RNA (lncRNA) LINC01551, wherein adjacent to or surrounding includes base positions within 20, 40, 50, 75, 100, or 150 base positions 5′ and/or 3′. In certain embodiments, the adjacent to or surrounding base positions are within 20 base positions 5′ and/or 3′. In certain embodiments, the adjacent to or surrounding base positions are within 40 base positions 5′ and/or 3′. In certain embodiments, the adjacent to or surrounding base positions are within 50 base positions 5′ and/or 3′. In certain embodiments, the adjacent to or surrounding base positions are within 75 base positions 5′ and/or 3′. In certain embodiments, the adjacent to or surrounding base positions are within 100 base positions 5′ and/or 3′. In certain embodiments, the adjacent to or surrounding base positions are within 150 base positions 5′ and/or 3′.

In some embodiments, the sequence is complementary to the target nucleic acid sequence at or adjacent (e.g., surrounding) to positions 100-300 or 360-560 or 730-970 or 780-1083 or 1228 to 1349 of long non-coding RNA (lncRNA) LINC02282 (e.g., reference sequence NR_026732.1 and NR_026731.1—merged exons). In some embodiments, the antisense oligonucleotide hybridizes to a target nucleic acid sequence at or adjacent (e.g., surrounding) to positions 100-300 or 360-560 or 730-970 or 780-1083 or 1228 to 1349 of long non-coding RNA (lncRNA) LINC01551. In certain embodiments, the antisense oligonucleotide hybridizes to a target nucleic acid sequence at or adjacent (e.g., surrounding) to the positions provided in Table 3 or 4 for long non-coding RNA (lncRNA) LINC02282. In certain embodiments, the antisense oligonucleotide hybridizes to a target nucleic acid sequence adjacent (e.g., surrounding) to the positions provided in Table 3 for long non-coding RNA (lncRNA) LINC02282, wherein adjacent to or surrounding includes base positions within 20, 40, 50, 75, 100, or 150 base positions 5′ and/or 3′. In certain embodiments, the antisense oligonucleotide hybridizes to a target nucleic acid sequence adjacent (e.g., surrounding) to the positions provided in Table 4 for long non-coding RNA (lncRNA) LINC02282, wherein adjacent to or surrounding includes base positions within 20, 40, 50, 75, 100, or 150 base positions 5′ and/or 3′. In certain embodiments, the adjacent to or surrounding base positions are within 20 base positions 5′ and/or 3′. In certain embodiments, the adjacent to or surrounding base positions are within 40 base positions 5′ and/or 3′. In certain embodiments, the adjacent to or surrounding base positions are within 50 base positions 5′ and/or 3′. In certain embodiments, the adjacent to or surrounding base positions are within 75 base positions 5′ and/or 3′. In certain embodiments, the adjacent to or surrounding base positions are within 100 base positions 5′ and/or 3′. In certain embodiments, the adjacent to or surrounding base positions are within 150 base positions 5′ and/or 3′.

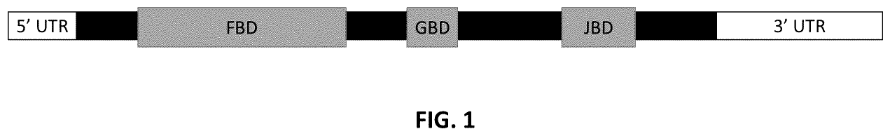

In certain instances, targeting (e.g. hybridization) to the lncRNA increases FOXG1 expression. In certain instances, targeting (e.g. hybridization) to the lncRNA prevents lncRNA-mediated down regulation of FOXG1 by promoting the degradation of the lncRNA In certain instances, targeting (e.g. hybridization) to the lncRNA prevents lncRNA-mediated down regulation of FOXG1 by promoting the degradation of the lncRNA. Accordingly, in some embodiments, hybridization of the sequence of the antisense oligonucleotide to the target nucleic acid sequence increases degradation of the lncRNA. In some embodiments, hybridization of the sequence of the antisense oligonucleotide to the target nucleic acid sequence increases expression of FOXG1. In certain embodiments, expression of FOXG1 is mRNA expression. In certain embodiments, expression of FOXG1 is protein expression. Such ASOs are suitable for use in the methods described herein. FIG. 1 shows a diagram of the FOXG1 mRNA transcript. TABLE 1 discloses sequences and antisense oligonucleotides (ASOs) sequences for targeting to lncRNAs.

Compositions comprising one or more of the ASOs described herein are useful. In certain embodiments, combing two or more ASOs having a different sequence are used to increase FOXG1 expression in a cell. In certain embodiments, the compositions are a pharmaceutical composition.

In order to improve the pharmacodynamic, pharmacokinetic, and biodistribution properties of antisense oligonucleotides (ASOs), the antisense oligonucleotides can be designed and engineered to comprise one or more chemical modifications (e.g. a modified inter-nucleoside linker, a modified nucleoside, or a combination thereof). Accordingly, in some embodiments, the antisense oligonucleotide is a modified oligonucleotide. In some embodiments, the antisense oligonucleotide comprises one or more modifications. In certain embodiments, the modification comprises a modified inter-nucleoside linker, a modified nucleoside, or a combination thereof

Modified Inter-Nucleoside Linkers

Modification of the inter-nucleoside linker (i.e. backbone) can be utilized to increase pharmacodynamic, pharmacokinetic, and biodistribution properties. For example, inter-nucleoside linker modifications prevent or reduce degradation by cellular nucleases, thus increasing the pharmacokinetics and bioavailability of the antisense oligonucleotide. Generally, a modified inter-nucleoside linker includes any linker other than other than phosphodiester (PO) liners, that covalently couples two nucleosides together. In some embodiments, the modified inter-nucleoside linker increases the nuclease resistance of the antisense oligonucleotide compared to a phosphodiester linker. For naturally occurring antisense oligonucleotides, the inter-nucleoside linker includes phosphate groups creating a phosphodiester bond between adjacent nucleosides. Modified inter-nucleoside linkers are particularly useful in stabilizing antisense oligonucleotides for in vivo use and may serve to protect against nuclease cleavage.

In some embodiments, the antisense oligonucleotide comprises one or more inter-nucleoside linkers modified from the natural phosphodiester to a linker that is for example more resistant to nuclease attack. In some embodiments a certain region (e.g. the 5′ and/or 3′ region) or regions (e.g. the 5′ and 3′ regions) of the antisense oligonucleotide, or contiguous nucleotide comprises a modified inter-nucleoside linker. In certain embodiments, a 5′ region and 3′ region of the ASO comprise a modified linker. In certain embodiments, a 5′ region and 3′ region of the ASO comprise a modified linker, wherein the ASO comprises an unmodified region or segment between a 5′ modified region and 3′ modified region of the ASO. In some embodiments all of the inter-nucleoside linkers of the antisense oligonucleotide, or contiguous nucleotide sequence thereof, are modified. In some embodiments all of the inter-nucleoside linkers of the antisense oligonucleotide, or contiguous nucleotide sequence thereof, are nuclease resistant inter-nucleoside linkers. In some embodiments the inter-nucleoside linkage comprises sulphur (S), such as a phosphorothioate inter-nucleoside linkage.

In certain instances, phosphorothioate inter-nucleoside linkers are particularly useful due to nuclease resistance and improved pharmacokinetics. In some embodiments, one or more of the inter-nucleoside linkers of the antisense oligonucleotide, or contiguous nucleotide sequence thereof, comprise a phosphorothioate inter-nucleoside linker. In some embodiments, all of the inter-nucleoside linkers of the antisense oligonucleotide, or contiguous nucleotide sequence thereof, comprise a phosphorothioate inter-nucleoside linker.

### Modified Nucleosides

Modifications to the ribose sugar or nucleobase can also be utilized to increase pharmacodynamic, pharmacokinetic, and biodistribution properties. Similar to modifications of the inter-nucleoside linker, nucleoside modifications prevent or reduce degradation by cellular nucleases, thus increasing the pharmacokinetics and bioavailability of the antisense oligonucleotide. Generally, a modified nucleoside includes the introduction of one or more modifications of the sugar moiety or the nucleobase moiety.

The antisense oligonucleotides, as described, can comprise one or more nucleosides comprising a modified sugar moiety, wherein the modified sugar moiety is a modification of the sugar moiety when compared to the ribose sugar moiety found in deoxyribose nucleic acid (DNA) and RNA. Numerous nucleosides with modification of the ribose sugar moiety can be utilized, primarily with the aim of improving certain properties of oligonucleotides, such as affinity and/or nuclease resistance. Such modifications include those where the ribose ring structure is modified. These modifications include replacement with a hexose ring (HNA), a bicyclic ring having a biradicle bridge between the C2 and C4 carbons on the ribose ring (e.g. locked nucleic acids (LNA)), or an unlinked ribose ring which typically lacks a bond between the C2 and C3 carbons (e.g. UNA). Other sugar modified nucleosides include, for example, bicyclohexose nucleic acids or tricyclic nucleic acids. Modified nucleosides also include nucleosides where the sugar moiety is replaced with a non-sugar moiety, for example in the case of peptide nucleic acids (PNA), or morpholino nucleic acids.

Sugar modifications also include modifications made by altering the substituent groups on the ribose ring to groups other than hydrogen, or the 2′-OH group naturally found in DNA and RNA nucleosides. Substituents may, for example be introduced at the 2′, 3′, 4′ or 5′ positions. Nucleosides with modified sugar moieties also include 2′ modified nucleosides, such as 2′ substituted nucleosides. Indeed, much focus has been spent on developing 2′ substituted nucleosides, and numerous 2′ substituted nucleosides have been found to have beneficial properties when incorporated into oligonucleotides, such as enhanced nucleoside resistance and enhanced affinity. A 2′ sugar modified nucleoside is a nucleoside that has a substituent other than H or OH at the 2′ position (2′ substituted nucleoside) or comprises a 2′ linked biradicle, and includes 2′ substituted nucleosides and LNA (2′-4′ biradicle bridged) nucleosides. Examples of 2′ substituted modified nucleosides are 2′-O-alkyl-RNA, 2′-O-methyl-RNA, 2′-alkoxy-RNA, 2′-O-methoxyethyl-oligos (MOE), 2′-amino-DNA, 2′-Fluoro-RNA, and 2′-F-ANA nucleoside. In some embodiments, the antisense oligonucleotide comprises one or more modified sugars. In some embodiments, the antisense oligonucleotide comprises only modified sugars. In certain embodiments, the antisense oligo comprises greater than 10%, 25%, 50%, 75%, or 90% modified sugars. In some embodiments, the modified sugar is a bicyclic sugar. In some embodiments, the modified sugar comprises a 2′-O-methoxyethyl (MOE) group.

In some embodiments, the antisense oligonucleotide comprises both inter-nucleoside linker modifications and nucleoside modifications. In some embodiments a certain region (e.g. the 5′ and/or 3′ region) or regions (e.g. the 5′ and 3′ regions) of the ASO linker modifications and nucleoside modifications. In certain embodiments, a 5′ region and 3′ region of the ASO comprise a modified linker and nucleoside modifications. In certain embodiments, a 5′ region and 3′ region of the ASO comprise a modified linker and nucleoside modifications, wherein the ASO comprises an unmodified region or segment between a 5′ modified region and 3′ modified region of the ASO.

### Gapmers

Further provided herein are modified ASOs comprising that promote degradation of a target lncRNA, wherein such ASOs can be referred to as gapmers ASOs. In certain instances, a gapmer or gapped ASO refers to an oligomeric compound having two modified external regions and an unmodified internal or central region or segment. For example, a gapmer generally refers to and encompasses an antisense oligonucleotide which comprises a region of RNase H recruiting oligonucleotides (gap) flanked 5′ and 3′ by regions which comprise one or more affinity enhancing modified nucleosides (flanks or wings). Gapmer oligonucleotides are generally used to inhibit a target RNA in a cell, such as a inhibitory lncRNA, via an antisense mechanism (and may therefore also be called antisense gapmer oligonucleotides). Gapmer oligonucleotides generally comprise a region of at least about 5 contiguous nucleotides which are capable or recruiting RNaseH (gap region), such as a region of DNA nucleotides, e.g. 6-14 DNA nucleotides, flanked 5′ and 3′ by regions which comprise affinity enhancing modified nucleosides, such as LNA or 2′ substituted nucleotides. In some embodiments, the flanking regions may be 1-8 nucleotides in length.

A high affinity modified nucleoside generally includes and refers a a modified nucleotide which, when incorporated into the oligonucleotide enhances the affinity of the oligonucleotide for its complementary target, for example as measured by the melting temperature (T^{m}). A high affinity modified nucleoside of the present invention preferably results in an increase in melting temperature between +0.5 to +12° C., more preferably between +1.5 to +10° C. and most preferably between +3 to +8° C. per modified nucleoside. Hgh affinity modified nucleosides generally include include for example, many 2′ substituted nucleosides as well as locked nucleic acids (LNA).

In some embodiments, the parent and child oligonucleotides are gapmer oligonucleotides which comprise a central region of at least 5 or more contiguous nucleosides, such as at least 5 contiguous DNA nucleosides, and a 5′ wing region comprising of 1-6 high affinity nucleoside analogues, such as LNA nucleosides and a 3′ wing region comprising of 1-6 high affinity nucleoside analogues, such as LNA 1-6 nucleosides. An LNA gapmer oligonucleotide is an oligonucleotide which comprises at least one LNA nucleoside in the wing regions, and may for example comprise at least one LNA in both the 5′ and 3′ wing regions.

For example, in some embodiments, the three regions are a contiguous sequence with the sugar moieties of the external regions being different than the sugar moieties of the internal region and wherein the sugar moiety of a particular region is essentially the same. In certain embodiments, each a particular region has the same sugar moiety. In certain instances, the sugar moieties of the external regions are the same and the gapmer is considered a symmetric gapmer. In another instance, the sugar moiety used in the 5′-external region is different from the sugar moiety used in the 3′-external region, the gapmer is an asymmetric gapmer. In certain embodiments, the external regions are each independently 1, 2, 3, 4 or about 5 nucleotide subunits and comprise non-naturally occurring sugar moieties. In further embodiments, the internal region comprising β-D-2′-deoxyribonucleosides. In certain embodiments, the external regions each, independently, comprise from 1 to about 5 nucleotides having non-naturally occurring sugar moieties and the internal region comprises from 6 to 18 unmodified nucleosides. In further embodiments, the internal region or the gap generally comprises β-D-2′-deoxyribonucleosides but can comprise non-naturally occurring sugar moieties.

In some embodiments, the gapped oligomeric compounds comprise an internal region of β-D-2′-deoxyribonucleosides with one of the two external regions comprising tricyclic nucleosides as disclosed herein. In certain embodiments, the gapped oligomeric compounds comprise an internal region of β-D-2′-deoxyribonucleosides with both of the external regions comprising tricyclic nucleosides as provided herein. In certain embodiments, gapped oligomeric compounds are provided herein wherein all of the nucleotides comprise non-naturally occurring sugar moieties, as described herein.

Gapmer nucleobase sequences are also provided in TABLE 1 that encompasses SEQ ID NOs: 1-274. In some embodiments, the ASOs or gapmers described herein promote degradation of a lncRNA molecule. In certain embodiments, the degradation is RNAse dependent (e.g. RNase H) degradation.

Also provided herein are gapmer antisense oligonucleotides comprising a sequence that hybridizes to a target nucleic acid sequence of a long non-coding RNA (lncRNA), wherein the lncRNA reduces expression of FOXG1. In some embodiments, expression of FOXG1 is measured by FOXG1 mRNA expression. In some embodiments, expression of FOXG1 is measured by FOXG1 protein expression.

In some embodiments, the antisense oligonucleotide comprises a modification. In some embodiments, the modification comprises a modified inter-nucleoside linker, a modified nucleoside, or a combination thereof. In some embodiments, the antisense oligonucleotide comprises the modified inter-nucleoside linkage. In some embodiments, the sequence comprises a nucleobase sequence as set forth in any one of Table 3. In some embodiments, the antisense oligonucleotide comprises a nucleobase sequence as set forth in any one of Table 4. In some embodiments, the target nucleic acid sequence comprises one or more nucleobases complementary to a sequence selected from Table 3. In some embodiments, the target nucleic acid sequence comprises one or more nucleobases within or adjacent to any one of the reference positions selected from Table 3. In some embodiments, the target nucleic acid sequence comprises one or more nucleobases complementary to a sequence selected from Table 4. In some embodiments, the target nucleic acid sequence comprises one or more nucleobases within or adjacent to any one of the reference positions selected from Table 4.

In some embodiments, hybridization of the antisense oligonucleotide increases FOXG1 expression in a cell. In some embodiments, the FOXG1 expression is FOXG1 mRNA expression. In some embodiments, the FOXG1 mRNA expression is measured by a probe based quantification assay. In some embodiments, the long non-coding RNA (lncRNA) is FOXG1-AS1, long intergenic non-protein coding RNA 1551, long intergenic non-protein coding RNA 2282 (LINC02282), or a combination thereof

### Pharmaceutical Compositions

Further provided herein are pharmaceutical compositions comprising any of the disclosed antisense oligonucleotides and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the oligonucleotide is used in the pharmaceutically acceptable diluent at a concentration of 50-300 μM solution. In some embodiments, the oligonucleotide, as described, is administered at a dose of 10-1000 μg.

The antisense oligonucleotides or oligonucleotide conjugates of the disclosure may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

### Methods of Use

The antisense oligonucleotides (ASOs) provided herein are useful for targeting a lncRNA, wherein an antisense oligonucleotide increases FOXG1 expression in a cell (e.g. expression of a functional FOXG1 mRNA and/or protein). The antisense oligonucleotides targeting a lncRNAs, as decribed herein, are further useful in methods for increasing the expression and/or amount of functional FOXG1 in a cell (e.g. an amount of functional FOXG1 mRNA or protein). Accordingly, provided herein are methods of modulating expression of FOXG1 in a cell, comprising contacting the cell with a composition comprising an antisense oligonucleotide that hybridizes to a target nucleic acid sequence of a long non-coding RNA (lcRNA).

Further provided, are methods of treating or ameliorating a FOXG1 disease or disorder in an individual having, or at risk of having, the FOXG1 disease or disorder, comprising administering to the individual an antisense oligonucleotide, wherein the antisense oligonucleotide comprises a sequence that hybridizes to a target nucleic acid sequence of a long non-coding RNA (lcRNA).

Generally, cells of interest include neuronal cells and/or cells associated with the brain or brain development. In some embodiments, the cell is located in a brain of an individual. In some embodiments, the cell is a neural cell. In certain embodiments, the cell is a neuron, astrocyte, or fibroblast. In some embodiments, the individual is a human. In certain embodiments, the human is an unborn human. In some embodiments, the cell and/or individual comprises a mutated FOXG1 gene, reduced FOXG1 expression, or a FOXG1 deficiency. In some embodiments the individual has been diagnosed with or at risk of a FOCG1 disease or disorder. In some embodiments the FOXG1 disease or disorder is FOXG1 syndrome.

In some embodiments, the antisense oligonucleotide comprises a sequence that is complementary to the target nucleic acid sequence of a long non-coding RNA (lcRNA). In some embodiments, the long non-coding RNA (lcRNA) is located within 1 kilobases (kb), 2 kb, 5 kb, 8 kb, or 10 kb of a gene encoding FOXG1. In some embodiments, the long non-coding RNA (lcRNA) is FOXG1-AS1, long non-protein coding RNA 1551 (LINC01151), long intergenic non-protein coding RNA 2282 (LINC02282), or a combination thereof. In some embodiments, the antisense oligonucleotide comprises a nucleobase sequence as set forth in any one of SEQ ID NOs: 1-274. In some embodiments, hybridization of the antisense oligonucleotide to the target nucleic acid sequence increases degradation of the lncRNA. In some embodiments, hybridization of the antisense oligonucleotide to the target nucleic acid sequence increases expression of FOXG1. In certain embodiments, expression of FOXG1 is mRNA expression. In certain embodiments, expression of FOXG1 is protein expression.

Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman et al., Goodman and Gilman's The Pharmacological Basis of Therapeutics, McGraw-Hill, New York, N.Y., 2001; Gennaro, Remington: The Science and Practice of Pharmacy, Lippincott, Williams, and Wilkins, New York, N.Y., 2000; Avis, et al. (eds.), Pharmaceutical Dosage Forms: Parenteral Medications, Marcel Dekker, N Y, 1993; Lieberman, et al. (eds.), Pharmaceutical Dosage Forms: Tablets, Marcel Dekker, N Y, 1990; Lieberman, et al. (eds.) Pharmaceutical Dosage Forms: Disperse Systems, Inc., New York, N.Y., 2000).

Compositions comprising antisense oligonucleotides (ASOs), as disclosed herein, can be provided by by doses at intervals of, e.g., one day, one week, or 1-7 times per week. A specific dose protocol is one involving the maximal dose or dose frequency that avoids significant undesirable side effects.

The disclosed antisense oligonucleotides or pharmaceutical compositions thereof can be administered topically (such as, to the skin, inhalation, ophthalmic or otic) or enterally (such as, orally or through the gastrointestinal tract) or parenterally (such as, intravenous, subcutaneous, intra-muscular, intracerebral, intracerebroventricular or intrathecal). In some embodiments the antisense oligonucleotide or pharmaceutical compositions thereof are administered by a parenteral route including intravenous, intraarterial, subcutaneous, intraperitoneal or intramuscular injection or infusion, intrathecal or intracranial, e.g. intracerebral or intraventricular, administration. In some embodiments the active oligonucleotide or oligonucleotide conjugate is administered intravenously.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The term “FOXG1,” as used herein, generally refers to the gene and gene products that encode a member of the fork-head transcription factor family. The encoded protein, which functions as a transcriptional repressor, is highly expressed in neural tissues during brain development. Mutations at this locus have been associated with Rett syndrome and a diverse spectrum of neurodevelopmental disorders defined as part of the FOXG1 syndrome. Depending the context of its use, “FOXG1” can refer to the FOXG1 gene, a FOXG1 deoxyribonucleic acid molecule (DNA), a FOXG1 ribonucleic acid molecule (RNA), or a FOXG1 protein. The mRNA sequence of FOXG1 is described in “NM_005249.5→NP_005240.3 forkhead box protein G1” or “accession number NM_005249.5” or the mRNA encoded by “NCBI GENE ID: 2290”. A functional FOXG1 protein describes the wild-type or unmutated FOXG1 gene, mRNA, and/or protein. Generally, “FOXG1” refers to a functional ‘FOXG1” gene or gene product, having normal function/activity within a cell. Deletions or mutations or variants of FOXG1 are indicative of non-functional FOXG1 variants having reduced, inhibited, or ablated FOXG1 function. As disclosed herein, the compositions and methods disclosed herein are primarily concerned with modulating or increasing or restoring an amount of FOXG1 (i.e. functional FOXG1) in a cell and/or individual.

The term “oligonucleotide,” as used herein, generally refers to the as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the disclosure is man-made, and is chemically synthesized, and is typically purified or isolated. The oligonucleotide disclosed may comprise one or more modified nucleosides or nucleotides.

The term “antisense oligonucleotide,” as used herein, refers to oligonucleotides capable of modulating expression of a target gene by hybridizing to a target nucleic acid, in particular to a contiguous sequence on a target nucleic acid. Preferably, the antisense oligonucleotides of the present disclosure are single stranded. In some embodiments, the antisense oligonucleotide is single stranded.

The term modified oligonucleotide refers to an oligonucleotide comprising one or more sugar-modified nucleosides, modified nucleobases, and/or modified inter-nucleoside linkers.

The term “modified nucleoside” or “nucleoside modification,” as used herein, refers to nucleosides modified as compared to the equivalent DNA or RNA nucleoside by the introduction of one or more modifications of the sugar moiety or the (nucleo)base moiety. In some embodiments, the modified nucleoside comprise a modified sugar moiety. The term modified nucleoside may also be used herein interchangeably with the term “nucleoside analogue” or modified “units” or modified “monomers”.

The term “modified inter-nucleoside linkage” is refers to linkers other than phosphodiester (PO) linkers, that covalently couples two nucleosides together. Nucleotides with modified inter-nucleoside linkage are also termed “modified nucleotides”. In some embodiments, the modified inter-nucleoside linkage increases the nuclease resistance of the oligonucleotide compared to a phosphodiester linkage. For naturally occurring oligonucleotides, the inter-nucleoside linkage includes phosphate groups creating a phosphodiester bond between adjacent nucleosides. Modified inter-nucleoside linkers are particularly useful in stabilizing oligonucleotides for in vivo use and may serve to protect against nuclease cleavage at regions of DNA or RNA nucleosides.

The term “nucleobase” includes the purine (e.g. adenine and guanine) and pyrimidine (e.g. uracil, thymine and cytosine) moiety present in nucleosides and nucleotides which form hydrogen bonds in nucleic acid hybridization. The term nucleobase also encompasses modified nucleobases which may differ from naturally occurring nucleobases but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants.

A nucleobase moiety can be modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. In some embodiments, the cytosine nucleobases in a 5′cg3′ motif is 5-methyl cytosine.

The term “hybridizing” or “hybridizes” or “targets” or “binds” describes two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tm) defined as the temperature at which half of the oligonucleotides are duplexed with the target nucleic acid.

The oligonucleotide comprises a contiguous nucleotide region which is complementary to or hybridizes to a sub-sequence or region of the target nucleic acid molecule. The term “target sequence” as used herein refers to a sequence of nucleotides present in the target nucleic acid which comprises the nucleobase sequence which is complementary to the contiguous nucleotide region or sequence of the oligonucleotide of the disclosure. In some embodiments, the target sequence consists of a region on the target nucleic acid which is complementary to the contiguous nucleotide region or sequence of the oligonucleotide of the present disclosure. In some embodiments the target sequence is longer than the complementary sequence of a single oligonucleotide, and may, for example represent a preferred region of the target nucleic acid which may be targeted by several oligonucleotides of the present disclosure.

In some instances, the oligonucleotide comprises a contiguous nucleotide region of at least 10 nucleotides which is complementary to or hybridizes to a target sequence present in the target nucleic acid molecule. In some instances, the contiguous nucleotide region (and therefore the target sequence) comprises of at least 10 contiguous nucleotides, such as 11, 12, 13, 14, 15, 16, 17, 18, 19, 20, 21, 22, 23, 24, 25, 30 contiguous nucleotides, such as from 15-30, such as from 18-23 contiguous nucleotides.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “a therapeutically effective amount” of a compound of the present application refers to an amount of the compound of the present application that will elicit the biological or medical response of a subject, for example, reduction or inhibition of tumor cell proliferation, or ameliorate symptoms, alleviate conditions, slow or delay disease progression, or prevent a disease, etc. In one non-limiting embodiment, the term “a therapeutically effective amount” refers to the amount of a compound of the present application that, when administered to a subject, is effective to at least partially alleviate, inhibit, prevent and/or ameliorate a condition, or a disorder or a disease, or at least partially inhibit activity of a targeted enzyme or receptor.

As used in the specification and claims, the singular forms “a”, “an” and “the” include plural references unless the context clearly dictates otherwise. For example, the term “a sample” includes a plurality of samples, including mixtures thereof.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “a therapeutically effective amount” of a compound of the present application refers to an amount of the compound of the present application that will elicit the biological or medical response of a subject, for example, reduction or inhibition of tumor cell proliferation, or ameliorate symptoms, alleviate conditions, slow or delay disease progression, or prevent a disease, etc. In one non-limiting embodiment, the term “a therapeutically effective amount” refers to the amount of a compound of the present application that, when administered to a subject, is effective to at least partially alleviate, inhibit, prevent and/or ameliorate a condition, or a disorder or a disease, or at least partially inhibit activity of a targeted enzyme or receptor.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

### Exemplary Embodiments

Accordingly, provided herein are antisense oligonucleotides (ASOs), comprising a sequence that hybridizes to a target nucleic acid sequence of a long non-coding RNA (lncRNA). In some embodiments, the lncRNA regulates expression of FOXG1. In some embodiments, the lncRNA reduces expression of FOXG1 messenger RNA. In some embodiments, the lncRNA reduces transcription of FOXG1 messenger RNA molecule. In some embodiments, the lncRNA reduces expression of FOXG1 protein. In some embodiments, the lncRNA reduces translation of a FOXG1 protein molecule.

In some embodiments, provided is an antisense oligonucleotide of any of the preceding embodiments, wherein the antisense oligonucleotide comprises a modification. In some embodiments, provided is an antisense oligonucleotide of any of the preceding embodiments, wherein the modification comprises a modified inter-nucleoside linker, a modified nucleoside, or a combination thereof. In some embodiments, provided is an antisense oligonucleotide of any of the preceding embodiments, wherein the antisense oligonucleotide comprises a modified inter-nucleoside linkage. In some embodiments, provided is an antisense oligonucleotide of any of the preceding embodiments, wherein the modified inter-nucleoside linkage is a phosphorothioate inter-nucleoside linkage. In some embodiments, provided is an antisense oligonucleotide of any of the preceding embodiments, wherein the antisense oligonucleotide comprises a phosphodiester inter-nucleoside linkage. In some embodiments, provided is an antisense oligonucleotide of any of the preceding embodiments, wherein the antisense oligonucleotide comprises a modified nucleoside. In some embodiments, provided is an antisense oligonucleotide of any of the preceding embodiments, wherein the modified nucleoside comprises a modified sugar. In some embodiments, provided is an antisense oligonucleotide of any of the preceding embodiments, wherein the modified sugar is a bicyclic sugar. In some embodiments, provided is an antisense oligonucleotide of any of the preceding embodiments, wherein the modified sugar comprises a 2′-O-methoxyethyl group.

In some embodiments, provided is an antisense oligonucleotide of any of the preceding embodiments, wherein the sequence is complementary to the target nucleic acid sequence of a long non-coding RNA (lncRNA). In some embodiments, provided is an antisense oligonucleotide of any of the preceding embodiments, wherein the long non-coding RNA (lncRNA) is located within 1 kilobases (kb), 2 kb, 5 kb, 8 kb, or 10 kb of a gene encoding FOXG1. In some embodiments, provided is an antisense oligonucleotide of any of the preceding embodiments, wherein the long non-coding RNA (lncRNA) is FOXG1-AS1, long non-protein coding RNA 1551 (LINC01151), long intergenic non-protein coding RNA 2282 (LINC02282), or a combination thereof.

In some embodiments, provided is an antisense oligonucleotide of any of the preceding embodiments, wherein the sequence comprises a nucleobase sequence as set forth in any one of Table 3. In some embodiments, provided is an antisense oligonucleotide of any of the preceding embodiments, wherein the antisense oligonucleotide comprises a nucleobase sequence as set forth in any one of Table 4. In some embodiments, provided is an antisense oligonucleotide of any of the preceding embodiments, wherein the antisense oligonucleotide hybridizes to the target nucleic acid sequence comprising or adjacent to any one or more of the positions provided in Table 3. In some embodiments, provided is an antisense oligonucleotide of any of the preceding embodiments, wherein adjacent to any one or more of the positions provided in Table 3 comprises base positions within 20, 40, 50, 75, 100, or 150 base positions 5′ and/or 3′. In some embodiments, provided is an antisense oligonucleotide of any of the preceding embodiments, wherein the antisense oligonucleotide hybridizes to the target nucleic acid sequence comprising or adjacent to any one or more of the positions provided in Table 4, In some embodiments, provided is an antisense oligonucleotide of any of the preceding embodiments, wherein adjacent to any one or more of the positions provided in Table 4 comprises base positions within 20, 40, 50, 75, 100, or 150 base positions 5′ and/or 3′.

In some embodiments, provided is an antisense oligonucleotide of any of the preceding embodiments, wherein hybridization of the sequence of the antisense oligonucleotide to the target nucleic acid sequence increases degradation of the lncRNA. In some embodiments, provided is an antisense oligonucleotide of any of the preceding embodiments, wherein hybridization of the sequence of the antisense oligonucleotide to the target nucleic acid sequence increases expression of FOXG1. In some embodiments, provided is an antisense oligonucleotide of any of the preceding embodiments, wherein expression of FOXG1 is mRNA expression. In some embodiments, provided is an antisense oligonucleotide of any of the preceding embodiments, wherein expression of FOXG1 is protein expression. A composition comprising one or more of the antisense oligonucleotides of any of the preceding embodiments. A pharmaceutical composition comprising the antisense oligonucleotide of any of the preceding embodiments 3 and a pharmaceutically acceptable carrier or diluent.

Further provided are methods of modulating expression of FOXG1 in a cell, comprising contacting the cell with a composition comprising an antisense oligonucleotide that hybridizes to a target nucleic acid sequence of a long non-coding RNA (lncRNA). Also provided are methods of treating or ameliorating a FOXG1 disease or disorder in an individual having, or at risk of having, the FOXG1 disease or disorder, comprising administering to the individual an antisense oligonucleotide, wherein the antisense oligonucleotide comprises a sequence that hybridizes to a target nucleic acid sequence of a long non-coding RNA (lncRNA).

In some embodiments, provided is a method of any of the preceding embodiments, wherein the cell is a located in a brain of an individual. In some embodiments, provided is a method of any of the preceding embodiments, wherein the individual is a human. In some embodiments, provided is a method of any of the preceding embodiments, wherein the individual comprises reduced FOXG1 expression or a FOXG1 deficiency. In some embodiments, provided is a method of any of the preceding embodiments, wherein the individual has a FOXG1 disease or disorder. In some embodiments, provided is a method of any of the preceding embodiments, wherein the FOXG1 disease or disorder is FOXG1 syndrome.

In some embodiments, provided is a method of any of the preceding embodiments, wherein the antisense oligonucleotide comprises a sequence that is complementary to the target nucleic acid sequence of a long non-coding RNA (lncRNA).

In some embodiments, provided is a method of any of the preceding embodiments, wherein the long non-coding RNA (lncRNA) is located within 1 kilobases (kb), 2 kb, 5 kb, 8 kb, or 10 kb of a gene encoding FOXG1. In some embodiments, provided is a method of any of the preceding embodiments, wherein the long non-coding RNA (lncRNA) is FOXG1-AS1, long non-protein coding RNA 1551 (LINC01151), long intergenic non-protein coding RNA 2282 (LINC02282), or a combination thereof.

In some embodiments, provided is a method of any of the preceding embodiments, wherein the antisense oligonucleotide comprises a nucleobase sequence as set forth in any one of Table 3.

In some embodiments, provided is a method of any of the preceding embodiments, wherein the antisense oligonucleotide comprises a nucleobase sequence as set forth in any one of Table 4. In some embodiments, provided is a method of any of the preceding embodiments, wherein the antisense oligonucleotide hybridizes to the target nucleic acid sequence comprising or adjacent to any one or more of the positions provided in Table 3. In some embodiments, provided is a method of any of the preceding embodiments, wherein adjacent to any one or more of the positions provided in Table 3 comprises base positions within 20, 40, 50, 75, 100, or 150 base positions 5′ and/or 3′. In some embodiments, provided is a method of any of the preceding embodiments, wherein the antisense oligonucleotide hybridizes to the target nucleic acid sequence comprising or adjacent to any one or more of the positions provided in Table 4, In some embodiments, provided is a method of any of the preceding embodiments, wherein adjacent to any one or more of the positions provided in Table 4 comprises base positions within 20, 40, 50, 75, 100, or 150 base positions 5′ and/or 3′.

In some embodiments, provided is a method of any of the preceding embodiments, wherein hybridization of the antisense oligonucleotide to the target nucleic acid sequence increases degradation of the lncRNA. In some embodiments, provided is a method of any of the preceding embodiments, wherein hybridization of the antisense oligonucleotide to the target nucleic acid sequence increases expression of FOXG1. In some embodiments, provided is a method of any of the preceding embodiments, wherein expression of FOXG1 is mRNA expression. In some embodiments, provided is a method of any of the preceding embodiments, expression of FOXG1 is protein expression.

In some embodiments, provided is a method of any of the preceding embodiments, wherein the antisense oligonucleotide is configured as a gapmer. In some embodiments, provided is a method of any of the preceding embodiments, wherein the antisense oligonucleotide comprises at least one modified inter-nucleoside linkage. In some embodiments, provided is a method of any of the preceding embodiments, wherein the modified inter-nucleoside linkage is a phosphorothioate inter-nucleoside linkage. In some embodiments, provided is a method of any of the preceding embodiments, wherein the antisense oligonucleotide comprises at least one phosphodiester inter-nucleoside linkage. In some embodiments, provided is a method of any of the preceding embodiments, wherein the antisense oligonucleotide comprises a modified nucleoside. In some embodiments, provided is a method of any of the preceding embodiments, the modified nucleoside comprises a modified sugar. In some embodiments, provided is a method of any of the preceding embodiments, the modified sugar is a bicyclic sugar. In some embodiments, provided is a method of any of the preceding embodiments, wherein the modified sugar comprises a 2′-O-methoxyethyl group.

In some embodiments, provided is a method of any of the preceding embodiments, wherein modulating expression comprises increasing expression of a FOXG1 protein in the cell.

In some embodiments, provided is a method of any of the preceding embodiments, wherein modulating expression comprises increasing translation of a FOXG1 protein in the cell.

In some embodiments, provided is a method of any of the preceding embodiments, wherein the antisense oligonucleotide is administered to the individual by intrathecal injection, intracerebroventricular injection, inhalation, parenteral injection or infusion, or orally.

### EXAMPLES

The following examples are included for illustrative purposes only and are not intended to limit the scope of the present disclosure.

### Example 1: Design and Selection of ASOs

Antisense oligonucleotides (“ASOs” or “oligos”) against the human FOXG1-AS1, LINC01151, and LINC02282 mRNAs were chosen as follows. Twenty-mer (“20mer”) nucleotide subsequences that were reverse-complementary to the lncRNA targets FOXG1-AS1 (NR_125758.1), LINC01551 (NR_026732.1 and NR_026731.1—merged exons) LINC02282 (NR_135255.1) were assembled. Thermal and sequence characteristics were then used to initially subset the oligos as follows:

Different characteristics were used in the initial selection step (above). In the above, Tₘ=Melting temperature of hybridization; Tₕₐᵢᵣₚᵢₙ=temperature of hairpin formation; T_{homodimer}=temperature of homodimer formation, as predicted by the Biopython software package (http://biopython.org). These selected 20mers were then further selected for specificity via sequence alignment to the complete human RefSeq unspliced transcriptome (downloaded Mar. 26, 2020). Alignment was conducted using the FASTA software suite (https://fasta.bioch.virginia.edu/fasta/fasta_list.html).

TABLE 1
Antisense oligonucleotides targeting lncRNA

| OligoID (Target) | Sequence |
|---|---|
| NR_125758.1_60-79_as | GGTATGTTTCGTGCCCATGT |
| NR_125758.1_62-81_as | GTGGTATGTTTCGTGCCCAT |
| NR_125758.1_63-82_as | TGTGGTATGTTTCGTGCCCA |
| NR_125758.1_64-83_as | ATGTGGTATGTTTCGTGCCC |
| NR_125758.1_65-84_as | AATGTGGTATGTTTCGTGCC |
| NR_125758.1_66-85_as | AAATGTGGTATGTTTCGTGC |
| NR_125758.1_67-86_as | AAAATGTGGTATGTTTCGTG |
| NR_125758.1_68-87_as | TAAAATGTGGTATGTTTCGT |
| NR_125758.1_69-88_as | GTAAAATGTGGTATGTTTCG |
| NR_125758.1_70-89_as | CGTAAAATGTGGTATGTTTC |
| NR_125758.1_71-90_as | CCGTAAAATGTGGTATGTTT |
| NR_125758.1_72-91_as | TCCGTAAAATGTGGTATGTT |
| NR_125758.1_82-101_as | GGCTACATCCTCCGTAAAAT |
| NR_125758.1_115-134_as | TCATTTATGCTTCTCCACCT |
| NR_125758.1_116-135_as | TTCATTTATGCTTCTCCACC |
| NR_125758.1_117-136_as | TTTCATTTATGCTTCTCCAC |
| NR_125758.1_118-137_as | CTTTCATTTATGCTTCTCCA |
| NR_125758.1_119-138_as | CCTTTCATTTATGCTTCTCC |
| NR_125758.1_120-139_as | GCCTTTCATTTATGCTTCTC |
| NR_125758.1_121-140_as | TGCCTTTCATTTATGCTTCT |
| NR_125758.1_122-141_as | GTGCCTTTCATTTATGCTTC |
| NR_125758.1_123-142_as | GGTGCCTTTCATTTATGCTT |
| NR_125758.1_124-143_as | AGGTGCCTTTCATTTATGCT |
| NR_125758.1_125-144_as | AAGGTGCCTTTCATTTATGC |
| NR_125758.1_155-174_as | AATTCTCTGTGCATCTTCTA |
| NR_125758.1_156-175_as | AAATTCTCTGTGCATCTTCT |
| NR_125758.1_157-176_as | GAAATTCTCTGTGCATCTTC |
| NR_125758.1_158-177_as | AGAAATTCTCTGTGCATCTT |
| NR_125758.1_167-186_as | CACAAGGTCAGAAATTCTCT |
| NR_125758.1_168-187_as | TCACAAGGTCAGAAATTCTC |
| NR_125758.1_169-188_as | GTCACAAGGTCAGAAATTCT |
| NR_125758.1_172-191_as | AACGTCACAAGGTCAGAAAT |
| NR_125758.1_205-224_as | TGGATGCCTCTGTATGGGAT |
| NR_125758.1_206-225_as | CTGGATGCCTCTGTATGGGA |
| NR_125758.1_208-227_as | ACCTGGATGCCTCTGTATGG |
| NR_125758.1_209-228_as | TACCTGGATGCCTCTGTATG |
| NR_125758.1_210-229_as | ATACCTGGATGCCTCTGTAT |
| NR_125758.1_211-230_as | AATACCTGGATGCCTCTGTA |
| NR_125758.1_212-231_as | AAATACCTGGATGCCTCTGT |
| NR_125758.1_213-232_as | GAAATACCTGGATGCCTCTG |
| NR_125758.1_214-233_as | GGAAATACCTGGATGCCTCT |
| NR_125758.1_268-287_as | ATTATAGACGAGTTGGCTCC |
| NR_125758.1_282-301_as | GCTGTTAGGAAGATATTATA |
| NR_125758.1_283-302_as | TGCTGTTAGGAAGATATTAT |
| NR_125758.1_284-303_as | CTGCTGTTAGGAAGATATTA |
| NR_125758.1_285-304_as | TCTGCTGTTAGGAAGATATT |
| NR_125758.1_286-305_as | TTCTGCTGTTAGGAAGATAT |
| NR_125758.1_287-306_as | GTTCTGCTGTTAGGAAGATA |
| NR_125758.1_288-307_as | GGTTCTGCTGTTAGGAAGAT |
| NR_125758.1_289-308_as | AGGTTCTGCTGTTAGGAAGA |
| NR_125758.1_290-309_as | CAGGTTCTGCTGTTAGGAAG |
| NR_125758.1_291-310_as | CCAGGTTCTGCTGTTAGGAA |
| NR_125758.1_292-311_as | CCCAGGTTCTGCTGTTAGGA |
| NR_125758.1_293-312_as | ACCCAGGTTCTGCTGTTAGG |
| NR_125758.1_296-315_as | GAGACCCAGGTTCTGCTGTT |
| NR_125758.1_297-316_as | TGAGACCCAGGTTCTGCTGT |
| NR_125758.1_414-433_as | CCGTACCTGTAGTTCCAGCT |
| NR_125758.1_416-435_as | TCCCGTACCTGTAGTTCCAG |
| NR_125758.1_417-436_as | TTCCCGTACCTGTAGTTCCA |
| NR 125758.1_418-437_as | TTTCCCGTACCTGTAGTTCC |
| NR_125758.1_419-438_as | TTTTCCCGTACCTGTAGTTC |
| NR_125758.1_420-439_as | GTTTTCCCGTACCTGTAGTT |
| NR_125758.1_421-440_as | AGTTTTCCCGTACCTGTAGT |
| NR_125758.1_476-495_as | CCGAAATTATTTTGTTAAAC |
| NR_125758.1_477-496_as | GCCGAAATTATTTTGTTAAA |
| NR_125758.1_478-497_as | AGCCGAAATTATTTTGTTAA |
| NR_125758.1_479-498_as | TAGCCGAAATTATTTTGTTA |
| NR 125758.1_480-499_as | ATAGCCGAAATTATTTTGTT |
| NR_125758.1_481-500_as | GATAGCCGAAATTATTTTGT |
| NR_125758.1_483-502_as | TTGATAGCCGAAATTATTTT |
| NR_125758.1_484-503_as | TTTGATAGCCGAAATTATTT |
| NR_125758.1_485-504_as | CTTTGATAGCCGAAATTATT |
| NR_125758.1_486-505_as | TCTTTGATAGCCGAAATTAT |
| NR_125758.1_488-507_as | GATCTTTGATAGCCGAAATT |
| NR_125758.1_489-508_as | TGATCTTTGATAGCCGAAAT |
| NR_125758.1_490-509_as | TTGATCTTTGATAGCCGAAA |
| NR_125758.1_491-510_as | CTTGATCTTTGATAGCCGAA |
| NR_125758.1_492-511_as | ACTTGATCTTTGATAGCCGA |
| NR_125758.1_493-512_as | CACTTGATCTTTGATAGCCG |
| NR_125758.1_494-513_as | CCACTTGATCTTTGATAGCC |
| NR_125758.1_498-517_as | TATCCCACTTGATCTTTGAT |
| NR_125758.1_499-518_as | TTATCCCACTTGATCTTTGA |
| NR_125758.1_501-520_as | ATTTATCCCACTTGATCTTT |
| NR_125758.1_502-521_as | AATTTATCCCACTTGATCTT |
| NR_125758.1_540-559_as | CCTCTATGGTATGCAAGGAG |
| NR_125758.1_552-571_as | ACCTCGACCTCTCCTCTATG |
| NR_125758.1_553-572_as | GACCTCGACCTCTCCTCTAT |
| NR_125758.1_625-644_as | GCTAGCAGACTCACACCACA |
| NR_125758.1_630-649_as | TCACGGCTAGCAGACTCACA |
| NR_125758.1_636-655_as | TGTCTCTCACGGCTAGCAGA |
| NR_125758.1_638-657_as | TCTGTCTCTCACGGCTAGCA |
| NR_125758.1_639-658_as | ATCTGTCTCTCACGGCTAGC |
| NR_125758.1_652-671_as | CCCTTTGTAATGCATCTGTC |
| NR_125758.1_653-672_as | TCCCTTTGTAATGCATCTGT |
| NR_125758.1_654-673_as | ATCCCTTTGTAATGCATCTG |
| NR_125758.1_655-674_as | CATCCCTTTGTAATGCATCT |
| NR_125758.1_656-675_as | CCATCCCTTTGTAATGCATC |
| NR_125758.1_657-676_as | TCCATCCCTTTGTAATGCAT |
| NR_125758.1_658-677_as | ATCCATCCCTTTGTAATGCA |
| NR_125758.1_659-678_as | AATCCATCCCTTTGTAATGC |
| NR_125758.1_660-679_as | AAATCCATCCCTTTGTAATG |
| NR_125758.1_661-680_as | TAAATCCATCCCTTTGTAAT |
| NR_125758.1_662-681_as | CTAAATCCATCCCTTTGTAA |
| NR_125758.1_663-682_as | ACTAAATCCATCCCTTTGTA |
| NR_125758.1_664-683_as | CACTAAATCCATCCCTTTGT |
| NR_125758.1_665-684_as | GCACTAAATCCATCCCTTTG |
| NR_125758.1_666-685_as | TGCACTAAATCCATCCCTTT |
| NR_125758.1_667-686_as | GTGCACTAAATCCATCCCTT |
| NR_125758.1_668-687_as | AGTGCACTAAATCCATCCCT |
| NR_125758.1_719-738_as | GTTTTGTTTCATTGTTCACT |
| NR_125758.1_720-739_as | AGTTTTGTTTCATTGTTCAC |
| NR_125758.1_721-740_as | AAGTTTTGTTTCATTGTTCA |
| NR_125758.1_730-749_as | CTTGGGAAGAAGTTTTGTTT |
| NR_125758.1_731-750_as | GCTTGGGAAGAAGTTTTGTT |
| NR_125758.1_764-783_as | ATCTCTTCAAACTATGGCAC |
| NR_125758.1_765-784_as | CATCTCTTCAAACTATGGCA |
| NR_125758.1_768-787_as | TGCCATCTCTTCAAACTATG |
| NR_125758.1_769-788_as | ATGCCATCTCTTCAAACTAT |
| NR_125758.1_770-789_as | GATGCCATCTCTTCAAACTA |
| NR_125758.1_863-882_as | TTGTATAAACTGTTGTTGCA |
| NR_026732.1_NR_026731.1_ | GAAGCTGAAGTGGTGTTGGG |
| merge_75-94_as | |
| NR_026732.1_NR_026731.1_ | AGAAGCTGAAGTGGTGTTGG |
| merge_76-95_as | |
| NR_026732.1_NR_026731.1_ | CTTTTCCTCGGCATCCTTCG |
| merge_171-190_as | |
| NR_026732.1_NR_026731.1_ | CCTTTTCCTCGGCATCCTTC |
| merge_172-191_as | |
| NR_026732.1_NR_026731.1_ | TCCTTTTCCTCGGCATCCTT |
| merge_173-192_as | |
| NR_026732.1_NR_026731.1_ | ATCCTTTTCCTCGGCATCCT |
| merge_174-193_as | |
| NR_026732.1_NR_026731.1_ | TATCCTTTTCCTCGGCATCC |
| merge_175-194_as | |
| NR_026732.1_NR_026731.1_ | ATATCCTTTTCCTCGGCATC |
| merge_176-195_as | |
| NR_026732.1_NR_026731.1_ | GATATCCTTTTCCTCGGCAT |
| merge_177-196_as | |
| NR_026732.1_NR_026731.1_ | TGATATCCTTTTCCTCGGCA |
| merge_178-197_as | |
| NR_026732.1_NR_026731.1_ | CCGATGCTCTGGAATCTCAA |
| merge_428-447_as | |
| NR_026732.1_NR_026731.1_ | TCCGATGCTCTGGAATCTCA |
| merge_429-448_as | |
| NR_026732.1_NR_026731.1_ | CATCCGATGCTCTGGAATCT |
| merge_431-450_as | |
| NR_026732.1_NR_026731.1_ | TCATCCGATGCTCTGGAATC |
| merge_432-451_as | |
| NR_026732.1_NR_026731.1_ | TTCATCCGATGCTCTGGAAT |
| merge_433-452_as | |
| NR_026732.1_NR_026731.1_ | ACTACCCCTATGCACGTGAG |
| merge_521-540_as | |
| NR_026732.1_NR_026731.1_ | GTTCTTCCCCAAATGCCTTT |
| merge_962-981_as | |
| NR_026732.1_NR_026731.1_ | TGTTCTTCCCCAAATGCCTT |
| merge_963-982_as | |
| NR_026732.1_NR_026731.1_ | TTGTTCTTCCCCAAATGCCT |
| merge_964-983_as | |
| NR_026732.1_NR_026731.1_ | GTTGTTCTTCCCCAAATGCC |
| merge_965-984_as | |
| NR_026732.1_NR_026731.1_ | CGTTGTTCTTCCCCAAATGC |
| merge_966-985_as | |
| NR_026732.1_NR_026731.1_ | CTTTCTCTGGAGACACATCA |
| merge_1002-1021_as | |
| NR_026732.1_NR_026731.1_ | ACTTTCTCTGGAGACACATC |
| merge_1003-1022_as | |
| NR_026732.1_NR_026731.1_ | GTTGTTTGTTTGTTTGTTTT |
| merge_1039-1058_as | |
| NR_026732.1_NR_026731.1_ | TGTTGTTTGTTTGTTTGTTT |
| merge_1040-1059_as | |
| NR_026732.1_NR_026731.1_ | TTGTTGTTTGTTTGTTTGTT |
| merge_1041-1060_as | |
| NR_026732.1_NR_026731.1_ | GTTGTTGTTTGTTTGTTTGT |
| merge_1042-1061_as | |
| NR_026732.1_NR_026731.1_ | TGTTGTTGTTTGTTTGTTTG |
| merge_1043-1062_as | |
| NR_026732.1_NR_026731.1_ | TTGTTGTTGTTTGTTTGTTT |
| merge_1044-1063_as | |
| NR_026732.1_NR_026731.1_ | ATTGTTGTTGTTTGTTTGTT |
| merge_1045-1064_as | |
| NR_026732.1_NR_026731.1_ | TATTGTTGTTGTTTGTTTGT |
| merge_1046-1065_as | |
| NR_026732.1_NR_026731.1_ | TTATTGTTGTTGTTTGTTTG |
| merge_1047-1066_as | |
| NR_026732.1_NR_026731.1_ | GTTTATTGTTGTTGTTTGTT |
| merge_1049-1068_as | |
| NR_026732.1_NR_026731.1_ | TGTTTATTGTTGTTGTTTGT |
| merge_1050-1069_as | |
| NR_026732.1_NR_026731.1_ | TTGTTTATTGTTGTTGTTTG |
| merge_1051-1070_as | |
| NR_026732.1_NR_026731.1_ | GTTGTTTATTGTTGTTGTTT |
| merge_1052-1071_as | |
| NR_026732.1_NR_026731.1_ | AGTTGTTTATTGTTGTTGTT |
| merge_1053-1072_as | |
| NR_026732.1_NR_026731.1_ | AAGTTGTTTATTGTTGTTGT |
| merge_1054-1073_as | |
| NR_026732.1_NR_026731.1_ | AGTGGAATGAGTCAGCCCGA |
| merge_1548-1567_as | |
| NR_026732.1_NR_026731.1_ | AAGTGGAATGAGTCAGCCCG |
| merge_1549-1568_as | |
| NR_026732.1_NR_026731.1_ | AAAGTGGAATGAGTCAGCCC |
| merge_1550-1569_as | |
| NR_026732.1_NR_026731.1_ | CCTGCTGGATAGGAATTAAT |
| merge_2247-2266_as | |
| NR_026732.1_NR_026731.1_ | GCCTGCTGGATAGGAATTAA |
| merge_2248-2267_as | |
| NR_026732.1_NR_026731.1_ | TTAAAGCCTGCTGGATAGGA |
| merge_2253-2272_as | |
| NR_026732.1_NR_026731.1_ | TGTTAAAGCCTGCTGGATAG |
| merge_2255-2274_as | |
| NR_026732.1_NR_026731.1_ | TTGTTAAAGCCTGCTGGATA |
| merge_2256-2275_as | |
| NR_026732.1_NR_026731.1_ | TTTGTTAAAGCCTGCTGGAT |
| merge_2257-2276_as | |
| NR_026732.1_NR_026731.1_ | TTTTGTTAAAGCCTGCTGGA |
| merge_2258-2277_as | |
| NR_026732.1_NR_026731.1_ | TTTTTGTTAAAGCCTGCTGG |
| merge_2259-2278_as | |
| NR_026732.1_NR_026731.1_ | GTTTTTGTTAAAGCCTGCTG |
| merge_2260-2279_as | |
| NR_026732.1_NR_026731.1_ | AGTTTTTGTTAAAGCCTGCT |
| merge_2261-2280_as | |
| NR_026732.1_NR_026731.1_ | TAGTTTTTGTTAAAGCCTGC |
| merge_2262-2281_as | |
| NR_026732.1_NR_026731.1_ | TCTTTAGTAGCTTTCATGGC |
| merge_2319-2338_as | |
| NR_026732.1_NR_026731.1_ | CTGGCTTTTCTTTAGTAGCT |
| merge_2327-2346_as | |
| NR_026732.1_NR_026731.1_ | GCTGTTTCTGGCTTTTCTTT |
| merge_2334-2353_as | |
| NR_026732.1_NR_026731.1_ | CGCTGTTTCTGGCTTTTCTT |
| merge_2335-2354_as | |
| NR_026732.1_NR_026731.1_ | ACGCTGTTTCTGGCTTTTCT |
| merge_2336-2355_as | |
| NR_026732.1_NR_026731.1_ | TACGCTGTTTCTGGCTTTTC |
| merge_2337-2356_as | |
| NR_026732.1_NR_026731.1_ | TTACGCTGTTTCTGGCTTTT |
| merge_2338-2357_as | |
| NR_026732.1_NR_026731.1_ | CTTACGCTGTTTCTGGCTTT |
| merge_2339-2358_as | |
| NR_026732.1_NR_026731.1_ | TCTTACGCTGTTTCTGGCTT |
| merge_2340-2359_as | |
| NR_026732.1_NR_026731.1_ | TTCTTACGCTGTTTCTGGCT |
| merge_2341-2360_as | |
| NR_026732.1_NR_026731.1_ | ATTCTTACGCTGTTTCTGGC |
| merge_2342-2361_as | |
| NR_026732.1_NR_026731.1_ | CCTCGTCTCTGAATCATATT |
| merge_2372-2391_as | |
| NR_026732.1_NR_026731.1_ | CACAATAGTAGTGGCCTTGT |
| merge_2428-2447_as | |
| NR_026732.1_NR_026731.1_ | TCACAATAGTAGTGGCCTTG |
| merge_2429-2448_as | |
| NR_026732.1_NR_026731.1_ | TTCACAATAGTAGTGGCCTT |
| merge_2430-2449_as | |
| NR_026732.1_NR_026731.1_ | ATTCACAATAGTAGTGGCCT |
| merge_2431-2450_as | |
| NR_026732.1_NR_026731.1_ | CCATGTTGACTTAGTTGGTC |
| merge_2675-2694_as | |
| NR_026732.1_NR_026731.1_ | ACCATGTTGACTTAGTTGGT |
| merge_2676-2695_as | |
| NR_026732.1_NR_026731.1_ | GCTACCATGTCTGACTAATT |
| merge_2731-2750_as | |
| NR_026732.1_NR_026731.1_ | TGCTACCATGTCTGACTAAT |
| merge_2732-2751_as | |
| NR_026732.1_NR_026731.1_ | GTGCTACCATGTCTGACTAA |
| merge_2733-2752_as | |
| NR_026732.1_NR_026731.1_ | TGTGCTACCATGTCTGACTA |
| merge_2734-2753_as | |
| NR_026732.1_NR_026731.1_ | ATGTGCTACCATGTCTGACT |
| merge_2735-2754_as | |
| NR_026732.1_NR_026731.1_ | CATGTGCTACCATGTCTGAC |
| merge_2736-2755_as | |
| NR_026732.1_NR_026731.1_ | TGGGTGATATTTGGTTCCAA |
| merge_3554-3573_as | |
| NR_026732.1_NR_026731.1_ | CTGAGGAAATTGATGGTATA |
| merge_3673-3692_as | |
| NR_026732.1_NR_026731.1_ | ACTGAGGAAATTGATGGTAT |
| merge_3674-3693_as | |
| NR_026732.1_NR_026731.1_ | GACCGTACGAGGGAATTTTA |
| merge_3710-3729_as | |
| NR_026732.1_NR_026731.1_ | TGACCGTACGAGGGAATTTT |
| merge_3711-3730_as | |
| NR_026732.1_NR_026731.1_ | TTGACCGTACGAGGGAATTT |
| merge_3712-3731_as | |
| NR_026732.1_NR_026731.1_ | TTTGACCGTACGAGGGAATT |
| merge_3713-3732_as | |
| NR_026732.1_NR_026731.1_ | TTTTGACCGTACGAGGGAAT |
| merge_3714-3733_as | |
| NR_135255.1_89-108_as | GCCTTCTGTACTGTGATGGG |
| NR_135255.1_90-109_as | AGCCTTCTGTACTGTGATGG |
| NR_135255.1_91-110_as | AAGCCTTCTGTACTGTGATG |
| NR_135255.1_192-211_as | ATGTGTGGGATGTAGGTAGG |
| NR_135255.1_193-212_as | AATGTGTGGGATGTAGGTAG |
| NR_135255.1_194-213_as | AAATGTGTGGGATGTAGGTA |
| NR_135255.1_204-223_as | GGGACTCCTGAAATGTGTGG |
| NR_135255.1_230-249_as | CGTTCTGTGTTTTGTAGAAT |
| NR_135255.1_232-251_as | GTCGTTCTGTGTTTTGTAGA |
| NR_135255.1_233-252_as | GGTCGTTCTGTGTTTTGTAG |
| NR_135255.1_234-253_as | TGGTCGTTCTGTGTTTTGTA |
| NR_135255.1_235-254_as | ATGGTCGTTCTGTGTTTTGT |
| NR_135255.1_236-255_as | TATGGTCGTTCTGTGTTTTG |
| NR_135255.1_237-256_as | ATATGGTCGTTCTGTGTTTT |
| NR_135255.1_243-262_as | TGGCTCATATGGTCGTTCTG |
| NR 135255.1_244-263_as | GTGGCTCATATGGTCGTTCT |
| NR_135255.1_245-264_as | AGTGGCTCATATGGTCGTTC |
| NR_135255.1_246-265_as | AAGTGGCTCATATGGTCGTT |
| NR 135255.1_455-474_as | CTCAGTGACAGCTAGGTGGA |
| NR_135255.1_456-475_as | TCTCAGTGACAGCTAGGTGG |
| NR_135255.1_458-477_as | ATTCTCAGTGACAGCTAGGT |
| NR_135255.1_462-481_as | CCGAATTCTCAGTGACAGCT |
| NR_135255.1_586-605_as | CAATGCAGAGTTTCTATTAC |
| NR_135255.1_669-688_as | CCCATTCCCAGGATGTTAGA |
| NR_135255.1_670-689_as | TCCCATTCCCAGGATGTTAG |
| NR_135255.1_671-690_as | TTCCCATTCCCAGGATGTTA |
| NR_135255.1_672-691_as | CTTCCCATTCCCAGGATGTT |
| NR_135255.1_673-692_as | ACTTCCCATTCCCAGGATGT |
| NR_135255.1_674-693_as | TACTTCCCATTCCCAGGATG |
| NR_135255.1_675-694_as | TTACTTCCCATTCCCAGGAT |
| NR_135255.1_676-695_as | GTTACTTCCCATTCCCAGGA |
| NR_135255.1_677-696_as | TGTTACTTCCCATTCCCAGG |
| NR_135255.1_678-697_as | GTGTTACTTCCCATTCCCAG |
| NR_135255.1_679-698_as | AGTGTTACTTCCCATTCCCA |
| NR_135255.1_680-699_as | CAGTGTTACTTCCCATTCCC |
| NR_135255.1_690-709_as | CCACCGATCCCAGTGTTACT |
| NR_135255.1_763-782_as | TTTCCATTCCTCTCTTCCAT |
| NR_135255.1_764-783_as | CTTTCCATTCCTCTCTTCCA |
| NR_135255.1_766-785_as | GCCTTTCCATTCCTCTCTTC |
| NR_135255.1_767-786_as | TGCCTTTCCATTCCTCTCTT |
| NR_135255.1_768-787_as | TTGCCTTTCCATTCCTCTCT |
| NR 135255.1_769-788_as | TTTGCCTTTCCATTCCTCTC |
| NR_135255.1_770-789_as | TTTTGCCTTTCCATTCCTCT |
| NR_135255.1_771-790_as | CTTTTGCCTTTCCATTCCTC |
| NR_135255.1_772-791_as | TCTTTTGCCTTTCCATTCCT |
| NR_135255.1_773-792_as | TTCTTTTGCCTTTCCATTCC |
| NR_135255.1_833-852_as | TGCTGATGGTGGGACTTTTT |
| NR 135255.1_834-853_as | TTGCTGATGGTGGGACTTTT |
| NR_135255.1_835-854_as | TTTGCTGATGGTGGGACTTT |
| NR_135255.1_836-855_as | TTTTGCTGATGGTGGGACTT |
| NR_135255.1_837-856_as | CTTTTGCTGATGGTGGGACT |
| NR_135255.1_838-857_as | TCTTTTGCTGATGGTGGGAC |
| NR_135255.1_839-858_as | TTCTTTTGCTGATGGTGGGA |
| NR_135255.1_840-859_as | CTTCTTTTGCTGATGGTGGG |
| NR_135255.1_841-860_as | ACTTCTTTTGCTGATGGTGG |
| NR_135255.1_842-861_as | GACTTCTTTTGCTGATGGTG |
| NR_135255.1_843-862_as | AGACTTCTTTTGCTGATGGT |
| NR_135255.1_844-863_as | GAGACTTCTTTTGCTGATGG |
| NR_135255.1_845-864_as | AGAGACTTCTTTTGCTGATG |
| NR_135255.1_862-881_as | GCTGCTATTTTAGAGGAAGA |
| NR_135255.1_863-882_as | GGCTGCTATTTTAGAGGAAG |
| NR_135255.1_867-886_as | CTTTGGCTGCTATTTTAGAG |
| NR_135255.1_869-888_as | CTCTTTGGCTGCTATTTTAG |
| NR_135255.1_870-889_as | TCTCTTTGGCTGCTATTTTA |
| NR_135255.1_876-895_as | ATTTTCTCTCTTTGGCTGCT |
| NR_135255.1_978-997_as | GTTCAGAAATTGGGATTAAT |
| NR_135255.1_979-998_as | TGTTCAGAAATTGGGATTAA |
| NR_135255.1_981-1000_as | GCTGTTCAGAAATTGGGATT |
| NR_135255.1_982-1001_as | TGCTGTTCAGAAATTGGGAT |
| NR_135255.1_983-1002_as | ATGCTGTTCAGAAATTGGGA |
| NR_135255.1_984-1003_as | AATGCTGTTCAGAAATTGGG |
| NR_135255.1_993-1012_as | GCTAAGTAAAATGCTGTTCA |
| NR_135255.1_994-1013_as | TGCTAAGTAAAATGCTGTTC |
| NR_135255.1_1226-1245_as | TTTCCAACAGGCTCTCGTTT |
| NR_135255.1_1227-1246_as | CTTTCCAACAGGCTCTCGTT |
| NR_135255.1_1228-1247_as | CCTTTCCAACAGGCTCTCGT |
| NR 135255.1_1229-1248_as | TCCTTTCCAACAGGCTCTCG |
| NR 135255.1_1327-1346_as | GGTAGAATGGGAAAGGTTTT |
| NR 135255.1_1328-1347_as | GGGTAGAATGGGAAAGGTTT |
| NR_135255.1_1329-1348_as | TGGGTAGAATGGGAAAGGTT |
| NR_135255.1_1330-1349_as | CTGGGTAGAATGGGAAAGGT |
| NR 135255.1_1536-1555_as | GCACAAGTGGCAAAGCAAAA |
| NR_135255.1_1537-1556_as | TGCACAAGTGGCAAAGCAAA |
| NR_135255.1_1545-1564_as | AGATCTGTTGCACAAGTGGC |

### Example 2: Identification of ASOs that Increase FOXG1 Expression in a Cell

The MOE gapmer antisense oligonucleotides (ASOs) designed and selected in Example 1 were tested for the ability to increase FOXG1 expression in cells. In order to screen gapmer antisense oligonucleotides (ASOs), CCF-STTG1 cells were obtained from ATCC (ATCC in partnership with LGC Standards, Wesel, Germany, cat. #ATCC-CRL-1718), cultured in RPMI-1540 (#30-2001, ATCC in partnership with LGC Standards, Wesel, Germany), supplemented to contain 10% fetal calf serum (1248D, Biochrom GmbH, Berlin, Germany), and 100 U/ml Penicillin/100 μg/ml Streptomycin (A2213, Biochrom GmbH, Berlin, Germany). Cells were grown at 37° C. in an atmosphere with 5% CO₂ in a humidified incubator. For ASO transfection, CCF-STTG1 cells were seeded at a density of 15,000 cells/well into 96-well tissue culture plates (#655180, GBO, Germany).

In CCF-STTG1 cells, transfection of ASOs was carried out with Lipofectamine2000 (Invitrogen/Life Technologies, Karlsruhe, Germany) according to manufacturer's instructions for reverse transfection with 0.5 μL Lipofectamine2000 per well. The single dose screen was performed with ASOs in quadruplicates at 50 nM, with an ASO targeting AHSA1 (MOE-gapmer) and mock transfected cells as controls. ASOs were targeting one out of three lncRNAs expected to influence expression levels of FoxG1, so that FoxG1 mRNA expression was the readout. After 24 h of incubation (48 h incubation time resulted in high toxicity, visible in the rounding up of cells and low GapDH levels and was therefore neglected for analysis) with ASOs, medium was removed and cells were lysed in 150 μl Medium-Lysis Mixture (1 volume lysis mixture, 2 volumes cell culture medium) and then incubated at 53° C. for 30 minutes. Quantigene-Singleplex assay was performed according to manufacturer's instructions (ThermoFisher, Germany) with probesets to human FoxG1 and to GapDH for normalization. Luminescence was read using 1420 Luminescence Counter (WALLAC VICTOR Light, Perkin Elmer, Rodgau-Jügesheim, Germany) following 30 minutes incubation at RT in the dark.

In a subsequent experiment, 21 ASOs from the single dose screen were selected, which either produced promising results with regards to FoxG1 upregulation, or served as controls which had down-regulated FoxG1 in the initial screen. ASOs were transfected in three concentrations, namely 50 nM, 20 nM and 2 nM, whereas Ahsa1 at 50 nM and 2 nM and mock transfected cells served as controls.

The Ahsa1-ASO (one 2′-oMe and one MOE-modified) served at the same time as unspecific control for respective target mRNA expression and as a positive control to analyze transfection efficiency with regards to Ahsa1 mRNA level. By hybridization with an Ahsa1 probeset, the mock transfected wells served as controls for Ahsa1 mRNA level. Transfection efficiency for each 96-well plate and both doses in the dual dose screen were calculated by relating Ahsa1-level with Ahsa1-ASO (normalized to GapDH) to Ahsa1-level obtained with mock controls.

For each well, the target mRNA level was normalized to the respective GAPDH mRNA level. The activity of a given ASO was expressed as percent mRNA concentration of the respective target (normalized to GAPDH mRNA) in treated cells, relative to the target mRNA concentration (normalized to GAPDH mRNA) averaged across control wells (set as 100% target expression). mRNA expression was quantified using QuantiGene. Table 2 provides the Human FoxG1 QG2.0 probeset (Accession #NM_005249) and Human GapDH QG2.0 probeset (Accession #NM_002046). Oligosequences “CEs” and “LEs” are depicted without the proprietary parts of their sequences. Cross reactivity with the cyno sequence was obtained by adding additional probes.

TABLE 2
QuantiGene Probesets.

| Oligo name | Sequence 5′-3′ | Accession#, position & function |
|---|---|---|
| QG2_hsFoxG1_1 | ggccagcttggcccg | NM_005249.1334.1348.LE |
| QG2_hsFoxG1_2 | gcgcaccgcgcttgaa | NM_005249.1349.1364.LE |
| QG2_hsFoxG1 3 | gccggtggaggtgaggc | NM_005249.1365.1381.CE |
| QG2_hsFoxG1_4 | cgcggtccatgaaggtgag | NM_005249.1382.1400.LE |
| QG2_hsFoxG1_5 | gccagtagagggagccgg | NM_005249.1401.1418.LE |
| QG2_hsFoxG1_6 | gacaggaagggcgacatgg | NM_005249.1419.1437.BL |
| QG2_hsFoxG1_7 | gcgggggtggtgcagg | NM_005249.1438.1453.BL |
| QG2_hsFoxG1_8 | tgtaactcaaagtgctgctggc | NM_005249.1454.1475.CE |
| QG2_hsFoxG1_9 | gccgacgtggtgccgt | NM_005249.1476.1491.LE |
| QG2_hsFoxG1_10 | atggggggctggggtag | NM_005249.1492.1509.LE |
| QG2_hsFoxG1_11 | tcaacacggagctgtagggc | NM_005249.1510.1529.CE |
| QG2_hsFoxG1_12 | gttgcccagcgagttctgag | NM_005249.1530.1549.LE |
| QG2_hsFoxG1_13 | gcggtggagaaggagtggtt | NM_005249.1550.1569.LE |
| QG2_hsFoxG1_14 | ccacgctcaggccgttg | NM_005249.1570.1586.BL |
| QG2_hsFoxG1_15 | cccgttgaccagccggt | NM_005249.1587.1603.CE |
| QG2_hsFoxG1_16 | cgtggcgtacgggatctc | NM_005249.1604.1621.LE |
| QG2_hsFoxG1_17 | gcggccgtgaggtggtg | NM_005249.1622.1638.LE |
| QG2_hsFoxG1_18 | gaggcggctagcgcg | NM_005249.1639.1653.CE |
| QG2_hsFoxG1_19 | caggccgcagggcacc | NM_005249.1654.1669.LE |
| QG2_hsFoxG1_20 | ccagagcagggcaccga | NM_005249.1670.1686.LE |
| QG2_hsFoxG1_21 | caggggttgagggagtaggtc | NM_005249.1687.1707.CE |
| QG2_hsFoxG1_22 | gcgagcaggttgacggag | NM_005249.1708.1725.LE |
| QG2_hsFoxG1_23 | gaaaaagtaactggtctggccc | NM_005249.1726.1747.LE |
| QG2_hsFoxG1_24 | ggtgcgggacgtgggg | NM_005249.1748.1763.CE |
| QG2_hsFoxG1_25 | tgctctgcgaagtcattgacg | NM_005249.1764.1784.LE |
| QG2_hsFoxG1_26 | ggcgctcatggacgtgc | NM_005249.1785.1801.LE |
| QG2_hsFoxG1_27 | aggaggacgcggccct | NM_005249.1802.1817.CE |

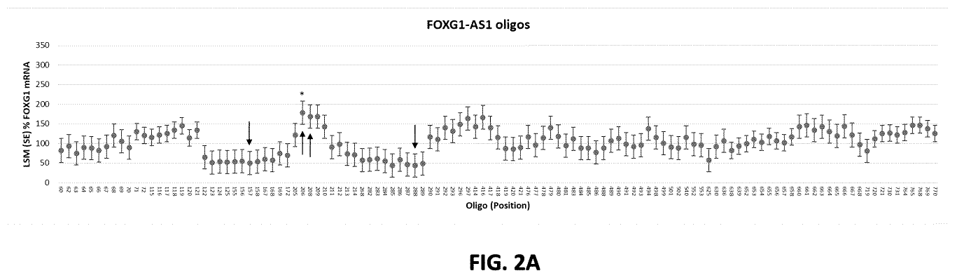

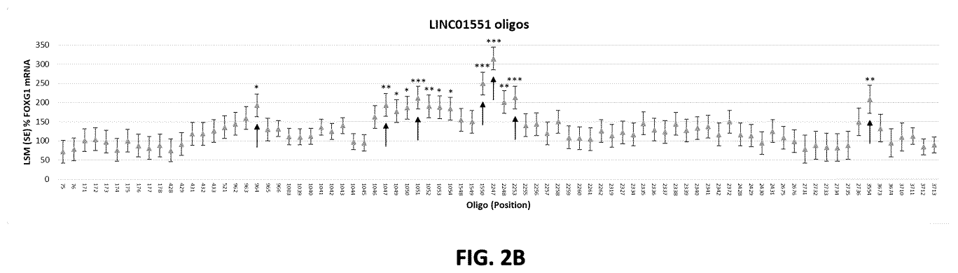

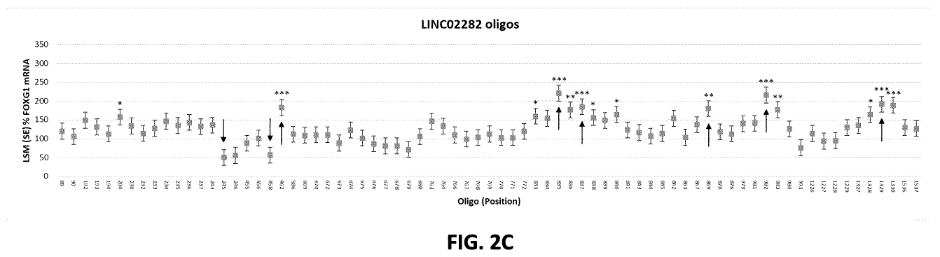

FIG. 2A-C shows that antisense gapmer oligonucleotides (ASOs) targeting long non-coding RNA (ncRNA) targets are abletin crease FOXG1 expression in cells. FIG. 2A-C provides the Least Square Mean Percent FOXG1 mRNA in CCF-STTG1 cells after treatment with 50 nM antisense oligos to knock down the FOXG1-AS1 (FIG. 2A), LINC01551 (FIG. 2B), or LINC02282 (FIG. 2C) lncRNA targets. Oligos are denoted by corresponding target mRNA position. FOXG1 mRNA was measured 24 hours post transfection. Stars indicate statistical significance relative to Mock and Control (non-targeting) oligos; *c, P<0.05; **, P<0.01; *** P<0.001. Arrows mark down- and up-regulatory oligos chosen for Dose Response Analysis. Tables 3 and 4 shows gapmer antisense oligonucleotides (ASOs) that increase FOXG1 expression, providing the Least Square Mean Percent FOXG1 mRNA in CCF-STTG1 cells, lncRNA, OligoID, sequence, position, and statistical significance (*, P<0.05; **, P<0.01; ***, P<0.001). Table 5 shows dose response data for gapmer antisense oligonucleotides (ASOs) at dose concentrations of 2, 20, and 50 nM, providing the target lncRNA, OligoID, response direction (“U”, up; “D”, down), the mean fold increase in FOXG1 expression, and standard error.

TABLE 3
Antisense oligonucleotides (ASOs) increasing FOXG1 expression

| | | | LSM | Significance |
|---|---|---|---|---|
| Target | OligoID | Position | (% FOXG1) | — |
| FOXG1-AS1 | NR_125758.1_68-87_as | 68 | 119.8358 | — |
| FOXG1-AS1 | NR_125758.1_69-88_as | 69 | 105.2692 | — |
| FOXG1-AS1 | NR_125758.1_71-90_as | 71 | 129.8516 | — |
| FOXG1-AS1 | NR_125758.1_72-91_as | 72 | 119.8393 | — |
| FOXG1-AS1 | NR_125758.1_115-134_as | 115 | 115.2154 | — |
| FOXG1-AS1 | NR_125758.1_116-135_as | 116 | 120.8962 | — |
| FOXG1-AS1 | NR_125758.1_117-136_as | 117 | 125.1282 | — |
| FOXG1-AS1 | NR_125758.1_118-137_as | 118 | 134.2027 | — |
| FOXG1-AS1 | NR_125758.1_119-138_as | 119 | 145.4411 | — |
| FOXG1-AS1 | NR_125758.1_120-139_as | 120 | 114.0783 | — |
| FOXG1-AS1 | NR_125758.1_121-140_as | 121 | 133.4272 | — |
| FOXG1-AS1 | NR_125758.1_205-224_as | 205 | 121.4066 | — |
| FOXG1-AS1 | NR_125758.1_206-225_as | 206 | 178.3451 | * |
| FOXG1-AS1 | NR_125758.1_208-227_as | 208 | 168.5946 | . |
| FOXG1-AS1 | NR_125758.1_209-228_as | 209 | 168.4846 | . |
| FOXG1-AS1 | NR_125758.1_210-229_as | 210 | 142.1497 | — |
| FOXG1-AS1 | NR_125758.1_290-309_as | 290 | 116.9382 | — |
| FOXG1-AS1 | NR_125758.1_291-310_as | 291 | 110.7735 | — |
| FOXG1-AS1 | NR_125758.1_292-311_as | 292 | 139.5714 | — |
| FOXG1-AS1 | NR_125758.1_293-312_as | 293 | 131.8477 | — |
| FOXG1-AS1 | NR_125758.1_296-315_as | 296 | 148.1122 | — |
| FOXG1-AS1 | NR_125758.1_297-316_as | 297 | 163.8187 | . |
| FOXG1-AS1 | NR_125758.1_414-433_as | 414 | 142.654 | — |
| FOXG1-AS1 | NR_125758.1_416-435_as | 416 | 166.4228 | . |
| FOXG1-AS1 | NR_125758.1_417-436_as | 417 | 139.3282 | — |
| FOXG1-AS1 | NR_125758.1_418-437_as | 418 | 115.4389 | — |
| FOXG1-AS1 | NR_125758.1_476-495_as | 476 | 116.6715 | — |
| FOXG1-AS1 | NR_125758.1_478-497_as | 478 | 113.534 | — |
| FOXG1-AS1 | NR_125758.1_479-498_as | 479 | 139.7402 | — |
| FOXG1-AS1 | NR_125758.1_480-499_as | 480 | 118.2394 | — |
| FOXG1-AS1 | NR_125758.1_483-502_as | 483 | 111.2582 | — |
| FOXG1-AS1 | NR_125758.1_489-508_as | 489 | 106.8954 | — |
| FOXG1-AS1 | NR_125758.1_490-509_as | 490 | 113.217 | — |
| FOXG1-AS1 | NR_125758.1_494-513_as | 494 | 137.5701 | — |
| FOXG1-AS1 | NR_125758.1_498-517_as | 498 | 114.7837 | — |
| FOXG1-AS1 | NR_125758.1_499-518_as | 499 | 100.4794 | — |
| FOXG1-AS1 | NR_125758.1_540-559_as | 540 | 115.324 | — |
| FOXG1-AS1 | NR_125758.1_636-655_as | 636 | 107.0681 | — |
| FOXG1-AS1 | NR_125758.1_653-672_as | 653 | 109.7666 | — |
| FOXG1-AS1 | NR_125758.1_654-673_as | 654 | 102.4918 | — |
| FOXG1-AS1 | NR_125758.1_655-674_as | 655 | 118.0954 | — |
| FOXG1-AS1 | NR_125758.1_656-675_as | 656 | 106.2447 | — |
| FOXG1-AS1 | NR_125758.1_657-676_as | 657 | 102.3042 | — |
| FOXG1-AS1 | NR_125758.1_658-677_as | 658 | 117.0233 | — |
| FOXG1-AS1 | NR_125758.1_660-679_as | 660 | 142.5884 | — |
| FOXG1-AS1 | NR_125758.1_661-680_as | 661 | 146.0999 | — |
| FOXG1-AS1 | NR_125758.1_662-681_as | 662 | 133.1997 | — |
| FOXG1-AS1 | NR_125758.1_663-682_as | 663 | 142.6474 | — |
| FOXG1-AS1 | NR_125758.1_664-683_as | 664 | 130.6147 | — |
| FOXG1-AS1 | NR_125758.1_665-684_as | 665 | 119.5423 | — |
| FOXG1-AS1 | NR_125758.1_666-685_as | 666 | 144.1696 | — |
| FOXG1-AS1 | NR_125758.1_667-686_as | 667 | 121.2138 | — |
| FOXG1-AS1 | NR_125758.1_720-739_as | 720 | 111.9995 | — |
| FOXG1-AS1 | NR_125758.1_721-740_as | 721 | 124.5812 | — |
| FOXG1-AS1 | NR_125758.1_730-749_as | 730 | 126.9396 | — |
| FOXG1-AS1 | NR_125758.1_731-750_as | 731 | 121.3403 | — |
| FOXG1-AS1 | NR_125758.1_764-783_as | 764 | 128.0099 | — |
| FOXG1-AS1 | NR_125758.1_765-784_as | 765 | 145.519 | — |
| FOXG1-AS1 | NR_125758.1_768-787_as | 768 | 146.7117 | — |
| FOXG1-AS1 | NR_125758.1_769-788_as | 769 | 137.4396 | — |
| FOXG1-AS1 | NR_125758.1_770-789_as | 770 | 124.9839 | — |
| LINC01551 | NR_026732.1_NR_026731..1_merge_171-190_as | 171 | 101.2436 | — |
| LINC01551 | NR_026732.1_NR_026731..1_merge_172-191_as | 172 | 103.6003 | — |
| LINC01551 | NR_026732.1_NR_026731..1_merge_431-450_as | 431 | 118.3662 | — |
| LINC01551 | NR_026732.1_NR_026731..1_merge_432-451_as | 432 | 117.9914 | — |
| LINC01551 | NR_026732.1_NR_026731..1_merge_433-452_as | 433 | 125.6478 | — |
| LINC01551 | NR_026732.1_NR_026731..1_merge_521-540_as | 521 | 135.4429 | — |
| LINC01551 | NR_026732.1_NR_026731..1_merge_962-981_as | 962 | 144.108 | — |
| LINC01551 | NR_026732.1_NR_026731..1_merge_963-982_as | 963 | 159.1389 | — |
| LINC01551 | NR_026732.1_NR_026731..1_merge_964-983_as | 964 | 192.7082 | ** |
| LINC01551 | NR_026732.1_NR_026731..1_merge_965-984_as | 965 | 129.34 | — |
| LINC01551 | NR_026732.1_NR_026731..1_merge_966-985_as | 966 | 131.5178 | — |
| LINC01551 | NR_026732.1_NR_026731..1_merge_1003-1022_as | 1003 | 111.0948 | — |
| LINC01551 | NR_026732.1_NR_026731..1_merge_1039-1058_as | 1039 | 109.8659 | — |
| LINC01551 | NR_026732.1_NR_026731..1_merge_1040-1059_as | 1040 | 111.6455 | — |
| LINC01551 | NR_026732.1_NR_026731..1_merge_1041-1060_as | 1041 | 135.8098 | — |
| LINC01551 | NR_026732.1_NR_026731..1_merge_1042-1061_as | 1042 | 124.2946 | — |
| LINC01551 | NR_026732.1_NR_026731..1_merge_1043-1062_as | 1043 | 139.7436 | — |
| LINC01551 | NR_026732.1_NR_026731..1_merge_1046-1065_as | 1046 | 162.4164 | . |
| LINC01551 | NR_026732.1_NR_026731..1_merge_1047-1066_as | 1047 | 193.177 | ** |
| LINC01551 | NR_026732.1_NR_026731..1_merge_1049-1068_as | 1049 | 176.6551 | * |
| LINC01551 | NR_026732.1_NR_026731..1_merge_1050-1069_as | 1050 | 186.5224 | * |
| LINC01551 | NR_026732.1_NR_026731..1_merge_1051-1070_as | 1051 | 211.9932 | *** |
| LINC01551 | NR_026732.1_NR_026731..1_merge_1052-1071_as | 1052 | 190.2748 | ** |
| LINC01551 | NR_026732.1_NR_026731..1_merge_1053-1072_as | 1053 | 187.2997 | * |
| LINC01551 | NR_026732.1_NR_026731..1_merge_1054-1073_as | 1054 | 184.0324 | * |
| LINC01551 | NR_026732.1_NR_026731..1_merge_1548-1567_as | 1548 | 154.7311 | — |
| LINC01551 | NR_026732.1_NR_026731..1_merge_1549-1568_as | 1549 | 149.9802 | — |
| LINC01551 | NR_026732.1_NR_026731..1_merge_1550-1569_as | 1550 | 249.4493 | *** |
| LINC01551 | NR_026732.1_NR_026731..1_merge_2247-2266_as | 2247 | 314.6698 | *** |
| LINC01551 | NR_026732.1_NR_026731..1_merge_2248-2267_as | 2248 | 200.632 | ** |
| LINC01551 | NR_026732.1_NR_026731..1_merge_2253-2272_as | 2253 | 212.562 | *** |
| LINC01551 | NR_026732.1_NR_026731..1_merge_2255-2274_as | 2255 | 140.2191 | — |
| LINC01551 | NR_026732.1_NR_026731..1_merge_2256-2275_as | 2256 | 143.2591 | — |
| LINC01551 | NR_026732.1_NR_026731..1_merge_2257-2276_as | 2257 | 119.5281 | — |
| LINC01551 | NR_026732.1_NR_026731..1_merge_2258-2277_as | 2258 | 149.5404 | — |
| LINC01551 | NR_026732.1_NR_026731..1_merge_2259-2278_as | 2259 | 108.7718 | — |
| LINC01551 | NR_026732.1_NR_026731..1_merge_2260-2279_as | 2260 | 107.0709 | — |
| LINC01551 | NR_026732.1_NR_026731..1_merge_2261-2280_as | 2261 | 104.2995 | — |
| LINC01551 | NR_026732.1_NR_026731..1_merge_2262-2281_as | 2262 | 125.7552 | — |
| LINC01551 | NR_026732.1_NR_026731..1_merge_2319-2338_as | 2319 | 112.9311 | — |
| LINC01551 | NR_026732.1_NR_026731..1_merge_2327-2346_as | 2327 | 122.0279 | — |
| LINC01551 | NR_026732.1_NR_026731..1_merge_2334-2353_as | 2334 | 116.5958 | — |
| LINC01551 | NR_026732.1_NR_026731..1_merge_2335-2354_as | 2335 | 145.574 | — |
| LINC01551 | NR_026732.1_NR_026731..1_merge_2336-2355_as | 2336 | 128.9508 | — |
| LINC01551 | NR_026732.1_NR_026731..1_merge_2337-2356_as | 2337 | 123.1395 | — |
| LINC01551 | NR_026732.1_NR_026731..1_merge_2338-2357_as | 2338 | 144.2022 | — |
| LINC01551 | NR_026732.1_NR_026731..1_merge_2339-2358_as | 2339 | 126.7695 | — |
| LINC01551 | NR_026732.1_NR_026731..1_merge_2340-2359_as | 2340 | 133.0967 | — |
| LINC01551 | NR_026732.1_NR_026731..1_merge_2341-2360_as | 2341 | 137.2337 | — |
| LINC01551 | NR_026732.1_NR_026731..1_merge_2342-2361_as | 2342 | 116.1773 | — |
| LINC01551 | NR_026732.1_NR_026731..1_merge_2372-2391_as | 2372 | 149.8658 | — |
| LINC01551 | NR_026732.1_NR_026731..1_merge_2428-2447_as | 2428 | 116.4803 | — |
| LINC01551 | NR_026732.1_NR_026731..1_merge_2429-2448_as | 2429 | 113.4778 | — |
| LINC01551 | NR_026732.1_NR_026731..1_merge_2431-2450_as | 2431 | 124.9652 | — |
| LINC01551 | NR_026732.1_NR_026731..1_merge_2675-2694_as | 2675 | 107.9292 | — |
| LINC01551 | NR_026732.1_NR_026731..1_merge_2736-2755_as | 2736 | 149.4117 | — |
| LINC01551 | NR_026732.1_NR_026731..1_merge_3554-3573_as | 3554 | 208.3008 | ** |
| LINC01551 | NR_026732.1_NR_026731..1_merge_3673-3692_as | 3673 | 132.79 | — |
| LINC01551 | NR_026732.1_NR_026731..1_merge_3710-3729_as | 3710 | 109.4747 | — |
| LINC01551 | NR_026732.1_NR_026731..1_merge_3711-3730_as | 3711 | 112.2256 | — |
| LINC02282 | NR_135255.1_89-108_as | 89 | 119.5063 | — |
| LINC02282 | NR_135255.1_90-109_as | 90 | 105.3562 | — |
| LINC02282 | NR_135255.1_192-211_as | 192 | 148.5745 | . |
| LINC02282 | NR_135255.1_193-212_as | 193 | 130.9477 | — |
| LINC02282 | NR_135255.1_194-213_as | 194 | 112.1581 | — |
| LINC02282 | NR_135255.1_204-223_as | 204 | 157.1731 | * |
| LINC02282 | NR_135255.1_230-249_as | 230 | 133.4666 | — |
| LINC02282 | NR_135255.1_232-251_as | 232 | 113.5322 | — |
| LINC02282 | NR_135255.1_233-252_as | 233 | 127.2036 | — |
| LINC02282 | NR_135255.1_234-253_as | 234 | 146.0912 | — |
| LINC02282 | NR_135255.1_235-254_as | 235 | 134.7058 | — |
| LINC02282 | NR_135255.1_236-255_as | 236 | 142.5579 | — |
| LINC02282 | NR_135255.1_237-256_as | 237 | 131.8185 | — |
| LINC02282 | NR_135255.1_243-262_as | 243 | 135.5674 | — |
| LINC02282 | NR_135255.1_456-475_as | 456 | 101.1669 | — |
| LINC02282 | NR_135255.1_462-481_as | 462 | 182.4739 | *** |
| LINC02282 | NR_135255.1_586-605_as | 586 | 111.6677 | — |
| LINC02282 | NR_135255.1_669-688_as | 669 | 108.6124 | — |
| LINC02282 | NR_135255.1_670-689_as | 670 | 109.8295 | — |
| LINC02282 | NR_135255.1_672-691_as | 672 | 109.6542 | — |
| LINC02282 | NR_135255.1_674-693_as | 674 | 122.3342 | — |
| LINC02282 | NR_135255.1_675-694_as | 675 | 100.8784 | — |
| LINC02282 | NR_135255.1_680-699_as | 680 | 105.2365 | — |
| LINC02282 | NR_135255.1_763-782_as | 763 | 145.577 | — |
| LINC02282 | NR_135255.1_764-783_as | 764 | 133.2802 | — |
| LINC02282 | NR_135255.1_766-785_as | 766 | 109.5801 | — |
| LINC02282 | NR_135255.1_768-787_as | 768 | 102.8706 | — |
| LINC02282 | NR_135255.1_769-788_as | 769 | 111.9311 | — |
| LINC02282 | NR_135255.1_770-789_as | 770 | 102.1801 | — |
| LINC02282 | NR_135255.1_771-790_as | 771 | 102.3618 | — |
| LINC02282 | NR_135255.1_772-791_as | 772 | 119.2151 | — |
| LINC02282 | NR_135255.1_833-852_as | 833 | 159.2383 | * |
| LINC02282 | NR_135255.1_834-853_as | 834 | 153.6097 | . |
| LINC02282 | NR_135255.1_835-854_as | 835 | 220.5611 | *** |
| LINC02282 | NR_135255.1_836-855_as | 836 | 176.0932 | ** |
| LINC02282 | NR_135255.1_837-856_as | 837 | 184.2671 | *** |
| LINC02282 | NR_135255.1_838-857_as | 838 | 155.0812 | * |
| LINC02282 | NR_135255.1_839-858_as | 839 | 147.9743 | . |
| LINC02282 | NR_135255.1_840-859_as | 840 | 163.6664 | * |
| LINC02282 | NR_135255.1_841-860_as | 841 | 122.8392 | — |
| LINC02282 | NR_135255.1_843-862_as | 843 | 115.8296 | — |
| LINC02282 | NR_135255.1_844-863_as | 844 | 106.7405 | — |
| LINC02282 | NR_135255.1_845-864_as | 845 | 113.1295 | — |
| LINC02282 | NR_135255.1_862-881_as | 862 | 153.6961 | . |
| LINC02282 | NR_135255.1_863-882_as | 863 | 103.0357 | — |
| LINC02282 | NR_135255.1_867-886_as | 867 | 136.8735 | — |
| LINC02282 | NR_135255.1_869-888_as | 869 | 179.7552 | ** |
| LINC02282 | NR_135255.1_870-889_as | 870 | 117.8833 | — |
| LINC02282 | NR_135255.1_876-895_as | 876 | 112.4724 | — |
| LINC02282 | NR_135255.1_979-998_as | 979 | 139.6044 | — |
| LINC02282 | NR_135255.1_981-1000_as | 981 | 140.7416 | — |
| LINC02282 | NR_135255.1_982-1001_as | 982 | 215.5858 | *** |
| LINC02282 | NR_135255.1_983-1002_as | 983 | 176.4842 | ** |
| LINC02282 | NR_135255.1_984-1003_as | 984 | 125.5474 | — |
| LINC02282 | NR_135255.1_1226-1245_as | 1226 | 113.2286 | — |
| LINC02282 | NR_135255.1_1229-1248_as | 1229 | 129.359 | — |
| LINC02282 | NR_135255.1_1327-1346_as | 1327 | 134.7263 | — |
| LINC02282 | NR_135255.1_1328-1347_as | 1328 | 163.2255 | * |
| LINC02282 | NR_135255.1_1329-1348_as | 1329 | 191.2834 | *** |
| LINC02282 | NR_135255.1_1330-1349_as | 1330 | 187.9057 | *** |
| LINC02282 | NR_135255.1_1536-1555_as | 1536 | 129.409 | — |
| LINC02282 | NR_135255.1_1537-1556_as | 1537 | 126.2619 | — |

TABLE 4
Antisense oligonucleotides (ASOs) increasing FOXG1 expression

| | | | LSM | |
|---|---|---|---|---|
| Target | Oligo ID | Position | (% FOXG1) | Significance |
| FOXG1-AS1 | NR_125758.1_206-225_as | 206 | 178.3451 | * |
| FOXG1-AS1 | NR_125758.1_208-227_as | 208 | 168.5946 | . |
| FOXG1-AS1 | NR_125758.1_209-228_as | 209 | 168.4846 | . |
| FOXG1-AS1 | NR_125758.1_297-316_as | 297 | 163.8187 | . |
| FOXG1-AS1 | NR_125758.1_416-435_as | 416 | 166.4228 | . |
| LINC01551 | NR_026732.1_NR_026731.1_merge_964-983_as | 964 | 192.7082 | ** |
| LINC01551 | NR_026732.1_NR_026731.1_merge_1046-1065_as | 1046 | 162.4164 | . |
| LINC01551 | NR_026732.1_NR_026731.1_merge_1047-1066_as | 1047 | 193.177 | ** |
| LINC01551 | NR_026732.1_NR_026731.1_merge_1049-1068_as | 1049 | 176.6551 | * |
| LINC01551 | NR_026732.1_NR_026731.1_merge_1050-1069_as | 1050 | 186.5224 | * |
| LINC01551 | NR_026732.1_NR_026731.1_merge_1051-1070_as | 1051 | 211.9932 | *** |
| LINC01551 | NR_026732.1_NR_026731.1_merge_1052-1071_as | 1052 | 190.2748 | ** |
| LINC01551 | NR_026732.1_NR_026731.1_merge_1053-1072_as | 1053 | 187.2997 | * |
| LINC01551 | NR_026732.1_NR_026731.1_merge_1054-1073_as | 1054 | 184.0324 | * |
| LINC01551 | NR_026732.1_NR_026731.1_merge_1550-1569_as | 1550 | 249.4493 | *** |
| LINC01551 | NR_026732.1_NR_026731.1_merge_2247-2266_as | 2247 | 314.6698 | *** |
| LINC01551 | NR_026732.1_NR_026731.1_merge_2248-2267_as | 2248 | 200.632 | ** |
| LINC01551 | NR_026732.1_NR_026731.1_merge_2253-2272_as | 2253 | 212.562 | *** |
| LINC01551 | NR_026732.1_NR_026731.1_merge_3554-3573_as | 3554 | 208.3008 | ** |
| LINC02282 | NR_135255.1_192-211_as | 192 | 148.5745 | . |
| LINC02282 | NR_135255.1_204-223_as | 204 | 157.1731 | * |
| LINC02282 | NR_135255.1_462-481_as | 462 | 182.4739 | *** |
| LINC02282 | NR_135255.1_833-852_as | 833 | 159.2383 | * |
| LINC02282 | NR_135255.1_834-853_as | 834 | 153.6097 | . |
| LINC02282 | NR_135255.1_835-854_as | 835 | 220.5611 | *** |
| LINC02282 | NR_135255.1_836-855_as | 836 | 176.0932 | ** |
| LINC02282 | NR_135255.1_837-856_as | 837 | 184.2671 | *** |
| LINC02282 | NR_135255.1_838-857_as | 838 | 155.0812 | * |
| LINC02282 | NR_135255.1_839-858_as | 839 | 147.9743 | . |
| LINC02282 | NR_135255.1_840-859_as | 840 | 163.6664 | * |
| LINC02282 | NR_135255.1_862-881_as | 862 | 153.6961 | . |
| LINC02282 | NR_135255.1_869-888_as | 869 | 179.7552 | ** |
| LINC02282 | NR_135255.1_982-1001_as | 982 | 215.5858 | *** |
| LINC02282 | NR_135255.1_983-1002_as | 983 | 176.4842 | ** |
| LINC02282 | NR_135255.1_1328-1347_as | 1328 | 163.2255 | * |
| LINC02282 | NR_135255.1_1329-1348_as | 1329 | 191.2834 | *** |
| LINC02282 | NR_135255.1_1330-1349_as | 1330 | 187.9057 | *** |

TABLE 5
Does-Repones Data for antisense oligonucleotides (ASOs)

| | | | | Dose | | |
|---|---|---|---|---|---|---|
| Target | Oligo ID | Position | Direction | (nM) | Mean | SEM |
| FOXG1- | NR_125758.1_157-176_as | 157 | D | 50 | 0.65221 | 0.007351 |
| AS1 | | | | 20 | 0.996095 | 0.030207 |
| | | | | 2 | 1.1133 | 0.147262 |
| | NR_125758.1_288-307_as | 288 | D | 50 | 0.599152 | 0.011942 |
| | | | | 20 | 0.833789 | 0.035904 |
| | | | | 2 | 1.384729 | 0.060105 |
| | NR_125758.1_206-225_as | 206 | U | 50 | 1.926113 | 0.03885 |
| | | | | 20 | 1.66569 | 0.020859 |
| | | | | 2 | 1.40545 | 0.023584 |
| | NR_125758.1_208-227_as | 208 | U | 50 | 1.408804 | 0.041198 |
| | | | | 20 | 1.286597 | 0.056838 |
| | | | | 2 | 1.277764 | 0.028176 |
| LINC01551 | NR_026732.1_NR_026731.1_merge_177-196_as | 177 | D | 50 | 0.588492 | 0.016346 |
| | | | | 20 | 0.660744 | 0.099281 |
| | | | | 2 | 1.224088 | 0.025018 |
| | NR_026732.1_NR_026731.1_merge_2733-2752_as | 2733 | D | 50 | 0.385547 | 0.009071 |
| | | | | 20 | 0.615311 | 0.00797 |
| | | | | 2 | 1.25387 | 0.037017 |
| | NR_026732.1_NR_026731.1_merge_964-983_as | 964 | U | 50 | 1.594955 | 0.042058 |
| | | | | 20 | 1.61334 | 0.0264 |
| | | | | 2 | 1.380871 | 0.00513 |
| | NR_026732.1_NR_026731.1_merge_1047-1066_as | 1047 | U | 50 | 1.107506 | 0.010679 |
| | | | | 20 | 1.169683 | 0.025297 |
| | | | | 2 | 1.314339 | 0.035626 |
| | NR_026732.1_NR_026731.1_merge_1051-1070_as | 1051 | U | 50 | 1.053834 | 0.021229 |
| | | | | 20 | 1.16509 | 0.025219 |
| | | | | 2 | 1.235187 | 0.084036 |
| | NR_026732.1_NR_026731.1_merge_1550-1569_as | 1550 | U | 50 | 0.650659 | 0.015247 |
| | | | | 20 | 0.818853 | 0.009704 |
| | | | | 2 | 1.130258 | 0.029575 |
| | NR_026732.1_NR_026731.1_merge_2247-2266_as | 2247 | U | 50 | 0.700006 | 0.008252 |
| | | | | 20 | 0.969129 | 0.008407 |
| | | | | 2 | 1.102238 | 0.022273 |
| | NR_026732.1_NR_026731.1_merge_2253-2272_as | 2253 | U | 50 | 0.965978 | 0.021046 |
| | | | | 20 | 1.063008 | 0.002901 |
| | | | | 2 | 1.151695 | 0.023599 |
| | NR_026732.1_NR_026731.1_merge_3554-3573_as | 3554 | U | 50 | 0.670076 | 0.007668 |
| | | | | 20 | 0.927593 | 0.025329 |
| | | | | 2 | 1.221273 | 0.033604 |
| LINC02282 | NR_135255.1_245-264_as | 245 | D | 50 | 0.602857 | 0.003982 |
| | | | | 20 | 0.697512 | 0.011445 |
| | | | | 2 | 1.220115 | 0.163149 |
| | NR_135255.1_458-477_as | 458 | D | 50 | 0.703038 | 0.015505 |
| | | | | 20 | 1.112802 | 0.082009 |
| | | | | 2 | 1.387779 | 0.024643 |
| | NR_135255.1_462-481_as | 462 | U | 50 | 1.036809 | 0.013467 |
| | | | | 20 | 1.203615 | 0.023542 |
| | | | | 2 | 1.304379 | 0.026297 |
| | NR_135255.1_835-854_as | 835 | U | 50 | 1.499956 | 0.048976 |
| | | | | 20 | 1.349677 | 0.023194 |
| | | | | 2 | 1.189141 | 0.035606 |
| | NR_135255.1_837-856_as | 837 | U | 50 | 1.388 | 0.019252 |
| | | | | 20 | 1.357614 | 0.016357 |
| | | | | 2 | 1.316696 | 0.013754 |
| | NR_135255.1_869-888_as | 869 | U | 50 | 0.920763 | 0.016696 |
| | | | | 20 | 1.089121 | 0.01285 |
| | | | | 2 | 1.37436 | 0.008316 |
| | NR_135255.1_982-1001_as | 982 | U | 50 | 1.696977 | 0.040296 |
| | | | | 20 | 1.420332 | 0.066222 |
| | | | | 2 | 1.298644 | 0.036198 |
| | NR_135255.1_1329-1348_as | 1329 | U | 50 | 1.732031 | 0.078662 |
| | | | | 20 | 1.387178 | 0.087631 |
| | | | | 2 | 1.20574 | 0.073535 |

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein may be employed in practicing the present disclosure. It is intended that the following claims define the scope of the present disclosure and that methods and structures within the scope of these claims and their equivalents be covered thereby.

SEQUENCES

| Number | Sequence |
|---|---|
| 1 | GGTATGTTTCGTGCCCATGT |
| 2 | GTGGTATGTTTCGTGCCCAT |
| 3 | TGTGGTATGTTTCGTGCCCA |
| 4 | ATGTGGTATGTTTCGTGCCC |
| 5 | AATGTGGTATGTTTCGTGCC |
| 6 | AAATGTGGTATGTTTCGTGC |
| 7 | AAAATGTGGTATGTTTCGTG |
| 8 | TAAAATGTGGTATGTTTCGT |
| 9 | GTAAAATGTGGTATGTTTCG |
| 10 | CGTAAAATGTGGTATGTTTC |
| 11 | CCGTAAAATGTGGTATGTTT |
| 12 | TCCGTAAAATGTGGTATGTT |
| 13 | GGCTACATCCTCCGTAAAAT |
| 14 | TCATTTATGCTTCTCCACCT |
| 15 | TTCATTTATGCTTCTCCACC |
| 16 | TTTCATTTATGCTTCTCCAC |
| 17 | CTTTCATTTATGCTTCTCCA |
| 18 | CCTTTCATTTATGCTTCTCC |
| 19 | GCCTTTCATTTATGCTTCTC |
| 20 | TGCCTTTCATTTATGCTTCT |
| 21 | GTGCCTTTCATTTATGCTTC |
| 22 | GGTGCCTTTCATTTATGCTT |
| 23 | AGGTGCCTTTCATTTATGCT |
| 24 | AAGGTGCCTTTCATTTATGC |
| 25 | AATTCTCTGTGCATCTTCTA |
| 26 | AAATTCTCTGTGCATCTTCT |
| 27 | GAAATTCTCTGTGCATCTTC |
| 28 | AGAAATTCTCTGTGCATCTT |
| 29 | CACAAGGTCAGAAATTCTCT |
| 30 | TCACAAGGTCAGAAATTCTC |
| 31 | GTCACAAGGTCAGAAATTCT |
| 32 | AACGTCACAAGGTCAGAAAT |
| 33 | TGGATGCCTCTGTATGGGAT |
| 34 | CTGGATGCCTCTGTATGGGA |
| 35 | ACCTGGATGCCTCTGTATGG |
| 36 | TACCTGGATGCCTCTGTATG |
| 37 | ATACCTGGATGCCTCTGTAT |
| 38 | AATACCTGGATGCCTCTGTA |
| 39 | AAATACCTGGATGCCTCTGT |
| 40 | GAAATACCTGGATGCCTCTG |
| 41 | GGAAATACCTGGATGCCTCT |
| 42 | ATTATAGACGAGTTGGCTCC |
| 43 | GCTGTTAGGAAGATATTATA |
| 44 | TGCTGTTAGGAAGATATTAT |
| 45 | CTGCTGTTAGGAAGATATTA |
| 46 | TCTGCTGTTAGGAAGATATT |
| 47 | TTCTGCTGTTAGGAAGATAT |
| 48 | GTTCTGCTGTTAGGAAGATA |
| 49 | GGTTCTGCTGTTAGGAAGAT |
| 50 | AGGTTCTGCTGTTAGGAAGA |
| 51 | CAGGTTCTGCTGTTAGGAAG |
| 52 | CCAGGTTCTGCTGTTAGGAA |
| 53 | CCCAGGTTCTGCTGTTAGGA |
| 54 | ACCCAGGTTCTGCTGTTAGG |
| 55 | GAGACCCAGGTTCTGCTGTT |
| 56 | TGAGACCCAGGTTCTGCTGT |
| 57 | CCGTACCTGTAGTTCCAGCT |
| 58 | TCCCGTACCTGTAGTTCCAG |
| 59 | TTCCCGTACCTGTAGTTCCA |
| 60 | TTTCCCGTACCTGTAGTTCC |
| 61 | TTTTCCCGTACCTGTAGTTC |
| 62 | GTTTTCCCGTACCTGTAGTT |
| 63 | AGTTTTCCCGTACCTGTAGT |
| 64 | CCGAAATTATTTTGTTAAAC |
| 65 | GCCGAAATTATTTTGTTAAA |
| 66 | AGCCGAAATTATTTTGTTAA |
| 67 | TAGCCGAAATTATTTTGTTA |
| 68 | ATAGCCGAAATTATTTTGTT |
| 69 | GATAGCCGAAATTATTTTGT |
| 70 | TTGATAGCCGAAATTATTTT |
| 71 | TTTGATAGCCGAAATTATTT |
| 72 | CTTTGATAGCCGAAATTATT |
| 73 | TCTTTGATAGCCGAAATTAT |
| 74 | GATCTTTGATAGCCGAAATT |
| 75 | TGATCTTTGATAGCCGAAAT |
| 76 | TTGATCTTTGATAGCCGAAA |
| 77 | CTTGATCTTTGATAGCCGAA |
| 78 | ACTTGATCTTTGATAGCCGA |
| 79 | CACTTGATCTTTGATAGCCG |
| 80 | CCACTTGATCTTTGATAGCC |
| 81 | TATCCCACTTGATCTTTGAT |
| 82 | TTATCCCACTTGATCTTTGA |
| 83 | ATTTATCCCACTTGATCTTT |
| 84 | AATTTATCCCACTTGATCTT |
| 85 | CCTCTATGGTATGCAAGGAG |
| 86 | ACCTCGACCTCTCCTCTATG |
| 87 | GACCTCGACCTCTCCTCTAT |
| 88 | GCTAGCAGACTCACACCACA |
| 89 | TCACGGCTAGCAGACTCACA |
| 90 | TGTCTCTCACGGCTAGCAGA |
| 91 | TCTGTCTCTCACGGCTAGCA |
| 92 | ATCTGTCTCTCACGGCTAGC |
| 93 | CCCTTTGTAATGCATCTGTC |
| 94 | TCCCTTTGTAATGCATCTGT |
| 95 | ATCCCTTTGTAATGCATCTG |
| 96 | CATCCCTTTGTAATGCATCT |
| 97 | CCATCCCTTTGTAATGCATC |
| 98 | TCCATCCCTTTGTAATGCAT |
| 99 | ATCCATCCCTTTGTAATGCA |
| 100 | AATCCATCCCTTTGTAATGC |
| 101 | AAATCCATCCCTTTGTAATG |
| 102 | TAAATCCATCCCTTTGTAAT |
| 103 | CTAAATCCATCCCTTTGTAA |
| 104 | ACTAAATCCATCCCTTTGTA |
| 105 | CACTAAATCCATCCCTTTGT |
| 106 | GCACTAAATCCATCCCTTTG |
| 107 | TGCACTAAATCCATCCCTTT |
| 108 | GTGCACTAAATCCATCCCTT |
| 109 | AGTGCACTAAATCCATCCCT |
| 110 | GTTTTGTTTCATTGTTCACT |
| 111 | AGTTTTGTTTCATTGTTCAC |
| 112 | AAGTTTTGTTTCATTGTTCA |
| 113 | CTTGGGAAGAAGTTTTGTTT |
| 114 | GCTTGGGAAGAAGTTTTGTT |
| 115 | ATCTCTTCAAACTATGGCAC |
| 116 | CATCTCTTCAAACTATGGCA |
| 117 | TGCCATCTCTTCAAACTATG |
| 118 | ATGCCATCTCTTCAAACTAT |
| 119 | GATGCCATCTCTTCAAACTA |
| 120 | TTGTATAAACTGTTGTTGCA |
| 121 | GAAGCTGAAGTGGTGTTGGG |
| 122 | AGAAGCTGAAGTGGTGTTGG |
| 123 | CTTTTCCTCGGCATCCTTCG |
| 124 | CCTTTTCCTCGGCATCCTTC |
| 125 | TCCTTTTCCTCGGCATCCTT |
| 126 | ATCCTTTTCCTCGGCATCCT |
| 127 | TATCCTTTTCCTCGGCATCC |
| 128 | ATATCCTTTTCCTCGGCATC |
| 129 | GATATCCTTTTCCTCGGCAT |
| 130 | TGATATCCTTTTCCTCGGCA |
| 131 | CCGATGCTCTGGAATCTCAA |
| 132 | TCCGATGCTCTGGAATCTCA |
| 133 | CATCCGATGCTCTGGAATCT |
| 134 | TCATCCGATGCTCTGGAATC |
| 135 | TTCATCCGATGCTCTGGAAT |
| 136 | ACTACCCCTATGCACGTGAG |
| 137 | GTTCTTCCCCAAATGCCTTT |
| 138 | TGTTCTTCCCCAAATGCCTT |
| 139 | TTGTTCTTCCCCAAATGCCT |
| 140 | GTTGTTCTTCCCCAAATGCC |
| 141 | CGTTGTTCTTCCCCAAATGC |
| 142 | CTTTCTCTGGAGACACATCA |
| 143 | ACTTTCTCTGGAGACACATC |
| 144 | GTTGTTTGTTTGTTTGTTTT |
| 145 | TGTTGTTTGTTTGTTTGTTT |
| 146 | TTGTTGTTTGTTTGTTTGTT |
| 147 | GTTGTTGTTTGTTTGTTTGT |
| 148 | TGTTGTTGTTTGTTTGTTTG |
| 149 | TTGTTGTTGTTTGTTTGTTT |
| 150 | ATTGTTGTTGTTTGTTTGTT |
| 151 | TATTGTTGTTGTTTGTTTGT |
| 152 | TTATTGTTGTTGTTTGTTTG |
| 153 | GTTTATTGTTGTTGTTTGTT |
| 154 | TGTTTATTGTTGTTGTTTGT |
| 155 | TTGTTTATTGTTGTTGTTTG |
| 156 | GTTGTTTATTGTTGTTGTTT |
| 157 | AGTTGTTTATTGTTGTTGTT |
| 158 | AAGTTGTTTATTGTTGTTGT |
| 159 | AGTGGAATGAGTCAGCCCGA |
| 160 | AAGTGGAATGAGTCAGCCCG |
| 161 | AAAGTGGAATGAGTCAGCCC |
| 162 | CCTGCTGGATAGGAATTAAT |
| 163 | GCCTGCTGGATAGGAATTAA |
| 164 | TTAAAGCCTGCTGGATAGGA |
| 165 | TGTTAAAGCCTGCTGGATAG |
| 166 | TTGTTAAAGCCTGCTGGATA |
| 167 | TTTGTTAAAGCCTGCTGGAT |
| 168 | TTTTGTTAAAGCCTGCTGGA |
| 169 | TTTTTGTTAAAGCCTGCTGG |
| 170 | GTTTTTGTTAAAGCCTGCTG |
| 171 | AGTTTTTGTTAAAGCCTGCT |
| 172 | TAGTTTTTGTTAAAGCCTGC |
| 173 | TCTTTAGTAGCTTTCATGGC |
| 174 | CTGGCTTTTCTTTAGTAGCT |
| 175 | GCTGTTTCTGGCTTTTCTTT |
| 176 | CGCTGTTTCTGGCTTTTCTT |
| 177 | ACGCTGTTTCTGGCTTTTCT |
| 178 | TACGCTGTTTCTGGCTTTTC |
| 179 | TTACGCTGTTTCTGGCTTTT |
| 180 | CTTACGCTGTTTCTGGCTTT |
| 181 | TCTTACGCTGTTTCTGGCTT |
| 182 | TTCTTACGCTGTTTCTGGCT |
| 183 | ATTCTTACGCTGTTTCTGGC |
| 184 | CCTCGTCTCTGAATCATATT |
| 185 | CACAATAGTAGTGGCCTTGT |
| 186 | TCACAATAGTAGTGGCCTTG |
| 187 | TTCACAATAGTAGTGGCCTT |
| 188 | ATTCACAATAGTAGTGGCCT |
| 189 | CCATGTTGACTTAGTTGGTC |
| 190 | ACCATGTTGACTTAGTTGGT |
| 191 | GCTACCATGTCTGACTAATT |
| 192 | TGCTACCATGTCTGACTAAT |
| 193 | GTGCTACCATGTCTGACTAA |
| 194 | TGTGCTACCATGTCTGACTA |
| 195 | ATGTGCTACCATGTCTGACT |
| 196 | CATGTGCTACCATGTCTGAC |
| 197 | TGGGTGATATTTGGTTCCAA |
| 198 | CTGAGGAAATTGATGGTATA |
| 199 | ACTGAGGAAATTGATGGTAT |
| 200 | GACCGTACGAGGGAATTTTA |
| 201 | TGACCGTACGAGGGAATTTT |
| 202 | TTGACCGTACGAGGGAATTT |
| 203 | TTTGACCGTACGAGGGAATT |
| 204 | TTTTGACCGTACGAGGGAAT |
| 205 | GCCTTCTGTACTGTGATGGG |
| 206 | AGCCTTCTGTACTGTGATGG |
| 207 | AAGCCTTCTGTACTGTGATG |
| 208 | ATGTGTGGGATGTAGGTAGG |
| 209 | AATGTGTGGGATGTAGGTAG |
| 210 | AAATGTGTGGGATGTAGGTA |
| 211 | GGGACTCCTGAAATGTGTGG |
| 212 | CGTTCTGTGTTTTGTAGAAT |
| 213 | GTCGTTCTGTGTTTTGTAGA |
| 214 | GGTCGTTCTGTGTTTTGTAG |
| 215 | TGGTCGTTCTGTGTTTTGTA |
| 216 | ATGGTCGTTCTGTGTTTTGT |
| 217 | TATGGTCGTTCTGTGTTTTG |
| 218 | ATATGGTCGTTCTGTGTTTT |
| 219 | TGGCTCATATGGTCGTTCTG |
| 220 | GTGGCTCATATGGTCGTTCT |
| 221 | AGTGGCTCATATGGTCGTTC |
| 222 | AAGTGGCTCATATGGTCGTT |
| 223 | CTCAGTGACAGCTAGGTGGA |
| 224 | TCTCAGTGACAGCTAGGTGG |
| 225 | ATTCTCAGTGACAGCTAGGT |
| 226 | CCGAATTCTCAGTGACAGCT |
| 227 | CAATGCAGAGTTTCTATTAC |
| 228 | CCCATTCCCAGGATGTTAGA |
| 229 | TCCCATTCCCAGGATGTTAG |
| 230 | TTCCCATTCCCAGGATGTTA |
| 231 | CTTCCCATTCCCAGGATGTT |
| 232 | ACTTCCCATTCCCAGGATGT |
| 233 | TACTTCCCATTCCCAGGATG |
| 234 | TTACTTCCCATTCCCAGGAT |
| 235 | GTTACTTCCCATTCCCAGGA |
| 236 | TGTTACTTCCCATTCCCAGG |
| 237 | GTGTTACTTCCCATTCCCAG |
| 238 | AGTGTTACTTCCCATTCCCA |
| 239 | CAGTGTTACTTCCCATTCCC |
| 240 | CCACCGATCCCAGTGTTACT |
| 241 | TTTCCATTCCTCTCTTCCAT |
| 242 | CTTTCCATTCCTCTCTTCCA |
| 243 | GCCTTTCCATTCCTCTCTTC |
| 244 | TGCCTTTCCATTCCTCTCTT |
| 245 | TTGCCTTTCCATTCCTCTCT |
| 246 | TTTGCCTTTCCATTCCTCTC |
| 247 | TTTTGCCTTTCCATTCCTCT |
| 248 | CTTTTGCCTTTCCATTCCTC |
| 249 | TCTTTTGCCTTTCCATTCCT |
| 250 | TTCTTTTGCCTTTCCATTCC |
| 251 | TGCTGATGGTGGGACTTTTT |
| 252 | TTGCTGATGGTGGGACTTTT |
| 253 | TTTGCTGATGGTGGGACTTT |
| 254 | TTTTGCTGATGGTGGGACTT |
| 255 | CTTTTGCTGATGGTGGGACT |
| 256 | TCTTTTGCTGATGGTGGGAC |
| 257 | TTCTTTTGCTGATGGTGGGA |
| 258 | CTTCTTTTGCTGATGGTGGG |
| 259 | ACTTCTTTTGCTGATGGTGG |
| 260 | GACTTCTTTTGCTGATGGTG |
| 261 | AGACTTCTTTTGCTGATGGT |
| 262 | GAGACTTCTTTTGCTGATGG |
| 263 | AGAGACTTCTTTTGCTGATG |
| 264 | GCTGCTATTTTAGAGGAAGA |
| 265 | GGCTGCTATTTTAGAGGAAG |
| 266 | CTTTGGCTGCTATTTTAGAG |
| 267 | CTCTTTGGCTGCTATTTTAG |
| 268 | TCTCTTTGGCTGCTATTTTA |
| 269 | ATTTTCTCTCTTTGGCTGCT |
| 270 | GTTCAGAAATTGGGATTAAT |
| 271 | TGTTCAGAAATTGGGATTAA |
| 272 | GCTGTTCAGAAATTGGGATT |
| 273 | TGCTGTTCAGAAATTGGGAT |
| 274 | ATGCTGTTCAGAAATTGGGA |
| 275 | AATGCTGTTCAGAAATTGGG |
| 276 | GCTAAGTAAAATGCTGTTCA |
| 277 | TGCTAAGTAAAATGCTGTTC |
| 278 | TTTCCAACAGGCTCTCGTTT |
| 279 | CTTTCCAACAGGCTCTCGTT |
| 280 | CCTTTCCAACAGGCTCTCGT |
| 281 | TCCTTTCCAACAGGCTCTCG |
| 282 | GGTAGAATGGGAAAGGTTTT |
| 283 | GGGTAGAATGGGAAAGGTTT |
| 284 | TGGGTAGAATGGGAAAGGTT |
| 285 | CTGGGTAGAATGGGAAAGGT |
| 286 | GCACAAGTGGCAAAGCAAAA |
| 287 | TGCACAAGTGGCAAAGCAAA |
| 288 | AGATCTGTTGCACAAGTGGC |

1.-103. (canceled)

104. An antisense oligonucleotide, comprising a sequence that hybridizes to a target nucleic acid sequence of a long non-coding RNA (lncRNA) or a pharmaceutically acceptable salt thereof, wherein the lncRNA regulates expression of FOXG1.

105. The antisense oligonucleotide of claim 104, wherein the sequence comprises a nucleobase sequence as set forth in any one of SEQ ID NOs: 8-9, SEQ ID NOs: 11-12, SEQ ID NOs: 14-20, SEQ ID NOs: 33-37, SEQ ID NOs: 51-60, SEQ ID NO: 64, SEQ ID NOs: 66-68, SEQ ID NO: 70, SEQ ID NOs: 75-76, SEQ ID NOs: 80-82, SEQ ID NO: 85, SEQ ID NO: 90, SEQ ID NOs: 94-99, SEQ ID NOs: 101-108, SEQ ID NOs: 111-119, SEQ ID NOs: 123-124, SEQ ID NOs: 133-141, SEQ ID NOs: 143-148, SEQ ID NOs: 151-186, SEQ ID NOs: 188-189, SEQ ID NOs: 196-198, SEQ ID NOs: 200-201, SEQ ID NOs: 205-206, SEQ ID NOs: 208-219, SEQ ID NO: 224, SEQ ID NOs: 226-229, SEQ ID NO: 231, SEQ ID NOs: 233-234, SEQ ID NO: 239, SEQ ID NOs: 241-243, SEQ ID NOs: 245-249, SEQ ID NOs: 251-259, SEQ ID NOs: 261-269, SEQ ID NOs: 271-275, SEQ ID NO: 278, or SEQ ID NOs: 281-287.

106. The antisense oligonucleotide of claim 104, wherein the sequence consists of a nucleobase sequence as set forth in any one of SEQ ID NOs: 8-9, SEQ ID NOs: 11-12, SEQ ID NOs: 14-20, SEQ ID NOs: 33-37, SEQ ID NOs: 51-60, SEQ ID NO: 64, SEQ ID NOs: 66-68, SEQ ID NO: 70, SEQ ID NOs: 75-76, SEQ ID NOs: 80-82, SEQ ID NO: 85, SEQ ID NO: 90, SEQ ID NOs: 94-99, SEQ ID NOs: 101-108, SEQ ID NOs: 111-119, SEQ ID NOs: 123-124, SEQ ID NOs: 133-141, SEQ ID NOs: 143-148, SEQ ID NOs: 151-186, SEQ ID NOs: 188-189, SEQ ID NOs: 196-198, SEQ ID NOs: 200-201, SEQ ID NOs: 205-206, SEQ ID NOs: 208-219, SEQ ID NO: 224, SEQ ID NOs: 226-229, SEQ ID NO: 231, SEQ ID NOs: 233-234, SEQ ID NO: 239, SEQ ID NOs: 241-243, SEQ ID NOs: 245-249, SEQ ID NOs: 251-259, SEQ ID NOs: 261-269, SEQ ID NOs: 271-275, SEQ ID NO: 278, or SEQ ID NOs: 281-287.

107. The antisense oligonucleotide of claim 104, wherein the antisense oligonucleotide comprises a nucleobase sequence as set forth in any one of SEQ ID NOs: 34-36, SEQ ID NO: 56, SEQ ID NO: 58, SEQ ID NO: 139, SEQ ID NO.: 151-158, SEQ ID NOs: 161-164, SEQ ID NO: 197, SEQ ID NO: 208, SEQ ID NO: 211, SEQ ID NO: 226, SEQ ID NOs: 251-258, SEQ ID NO: 264, SEQ ID NO: 267, SEQ ID NOs: 273-274, or SEQ ID NOs: 283-285.

108. The antisense oligonucleotide of claim 104, wherein the sequence consists of a nucleobase sequence as set forth in any one of SEQ ID NOs: 34-36, SEQ ID NO: 56, SEQ ID NO: 58, SEQ ID NO: 139, SEQ ID NOs: 151-158, SEQ ID NOs: 161-164, SEQ ID NO: 197, SEQ ID NO: 208, SEQ ID NO: 211, SEQ ID NO: 226, SEQ ID NOs: 251-258, SEQ ID NO: 264, SEQ ID NO: 267, SEQ ID NOs: 273-274, or SEQ ID NOs: 283-285.

109. The antisense oligonucleotide of claim 104, wherein the antisense oligonucleotide comprises a modification.

110. The antisense oligonucleotide of claim 109, wherein the antisense oligonucleotide comprises a modified inter-nucleoside linkage, optionally wherein the modified inter-nucleoside linkage is a phosphorothioate inter-nucleoside linkage or a phosphodiester inter-nucleoside linkage.

111. The antisense oligonucleotide of claim 109, wherein the antisense oligonucleotide is configured as a gapmer antisense oligonucleotide.

112. The antisense oligonucleotide of claim 109, wherein the antisense oligonucleotide comprises a modified nucleoside.

113. The antisense oligonucleotide of claim 112, wherein the modified nucleoside comprises a modified sugar, optionally wherein the modified sugar is a bicyclic sugar.

114. The antisense oligonucleotide of claim 113, wherein the modified sugar comprises a 2′-O-methoxyethyl group.

115. The antisense oligonucleotide of claim 104, wherein the long non-coding RNA (lncRNA) is FOXG1-AS1, long intergenic non-protein coding RNA 1551, long intergenic non-protein coding RNA 2282 (LINC02282), or a combination thereof.

116. A pharmaceutical composition comprising the antisense oligonucleotide of claim 104 and a pharmaceutically acceptable carrier or diluent.

117. A method of modulating expression of FOXG1 in a cell, comprising contacting the cell with a composition comprising an antisense oligonucleotide that hybridizes to a target nucleic acid sequence of a long non-coding RNA (lncRNA), wherein the lncRNA regulates expression of FOXG1.

118. The method of claim 117, wherein the cell is a located in a brain of an individual.

119. The method of claim 118, wherein the individual is a human.

120. The method of claim 118, wherein the individual comprises reduced FOXG1 expression or a FOXG1 deficiency.

121. The method of claim 118, wherein the individual has a FOXG1 disease or disorder.

122. The method of claim 121, wherein the FOXG1 disease or disorder is FOXG1 syndrome.

123. A method of treating or ameliorating a FOXG1 disease or disorder in an individual having, or at risk of having, the FOXG1 disease or disorder, comprising administering to the individual an antisense oligonucleotide, wherein the antisense oligonucleotide comprises a sequence that hybridizes to a target nucleic acid sequence of a long non-coding RNA (lncRNA), wherein the lncRNA regulates expression of FOXG1.
